(19) United States
(12) Patent Application Publication
(10) Pub. No.: US 2003/0232788 A1
(43) Pub. Date: Dec. 18, 2003

(54) (Substituted)acyl dipeptidyl inhibitors of the ICE/ced-3 family of cysteine proteases

(75) Inventors: Donald S. Karanewsky, Escondido, CA (US); Vincent J. Kalish, Annapolis, MD (US); Edward D. Robinson, San Diego, CA (US); Brett R. Ullman, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Idun Pharmaceuticals, Inc., San Diego, CA

(21) Appl. No.: 10/360,559

(22) Filed: Feb. 7, 2003

Related U.S. Application Data
(60) Provisional application No. 60/355,390, filed on Feb. 8, 2002.

(51) Int. Cl. A61K 38/04; A61K 31/675; A61K 31/5377; A61K 31/501; A61K 31/496; A61K 31/495; A61K 31/4709; A61K 31/422; A61K 31/401; C 07D 4 3/02; C07D 417/02

(52) U.S. Cl. 514/79; 514/247; 514/235.8; 514/235.2; 514/252.02; 514/310; 514/255.01; 514/400; 514/423; 514/397; 514/19; 514/616; 544/128; 544/141; 544/120; 544/114; 544/238; 544/363; 544/372; 544/366; 546/146; 548/533; 564/152

(57) ABSTRACT

This invention is directed to novel (substituted)acyl dipeptidyl ICE/ced-3 family inhibitor compounds. The invention is also directed to pharmaceutical compositions containing these compounds, as well as the use of such compositions in the treatment of patients suffering inflammatory, autoimmune and neurodegenerative diseases, for the prevention of ischemic injury, and for the preservation of organs that are to undergo a transplantation procedure.

### CROSS-REFERENCE TO RELATED APPLICATION

This application claims the benefit of U.S. Provisional Patent Application No. 60/355,390 filed Feb. 8, 2002, which provisional application is incorporated herein by reference in its entirety.

### BACKGROUND OF THE INVENTION

1. Field of the Invention

The present invention relates to novel classes of compounds that are inhibitors of interleukin-1β converting enzyme and related proteases (“ICE/ced-3 family of cysteine proteases”), as well as to pharmaceutical compositions comprising these compounds and to methods of using such pharmaceutical compositions.

2. Description of the Related Art

Interleukin 1 (“IL-1”) is a major pro-inflammatory and immunoregulatory protein that stimulates fibroblast differentiation and proliferation, the production of prostaglandins, collagenase and phospholipase by synovial cells and chondrocytes, basophil and eosinophil degranulation and neutrophil activation. Oppenheim, J. H. et al., Immunology Today, 7:45-56 (1986). As such, it is involved in the pathogenesis of chronic and acute inflammatory and autoimmune diseases. IL-1 is predominantly produced by peripheral blood monocytes as part of the inflammatory response. Mosely, B. S. et al., Proc. Nat. Acad. Sci., 84:4572-4576 (1987); Lonnemann, G. et al., Eur. J Immunol., 19:1531-1536 (1989).

IL-1β is synthesized as a biologically inactive precursor, proIL-1β. ProIL-1β is cleaved by a cysteine protease called interleukin-1β converting enzyme (“ICE”) between Asp-116 and Ala-117 to produce the biologically active C-terminal fragment found in human serum and synovial fluid. Sleath, P. R. et al., J. Biol. Chem., 265:14526-14528 (1992); A. D. Howard et al., J. Immunol., 147:2964-2969 (1991).

ICE is a cysteine protease localized primarily in monocytes. In addition to promoting the pro-inflammatory and immunoregulatory properties of IL-1β, ICE, and particularly its homologues, also appear to be involved in the regulation of cell death or apoptosis. Yuan, J. et al., Cell, 75:641-652 (1993); Miura, M. et al., Cell, 75:653-660 (1993); Nett-Giordalisi, M. A. et al., J. Cell Biochem., 17B:117 (1993). In particular, ICE or ICE/ced-3 homologues are thought to be associated with the regulation of apoptosis in neurogenerative diseases, such as Alzheimer's and Parkinson's disease. Marx, J. and M. Baringa, Science, 259:760-762 (1993); Gagliardini, V. et al., Science, 263:826-828 (1994).

Thus, disease states in which inhibitors of the ICE/ced-3 family of cysteine proteases may be useful as therapeutic agents include: infectious diseases, such as meningitis and salpingitis; septic shock, respiratory diseases; inflammatory conditions, such as arthritis, cholangitis, colitis, encephalitis, endocerolitis, hepatitis, pancreatitis and reperfusion injury, ischemic diseases such as the myocardial infarction, stroke and ischemic kidney disease; immune-based diseases, such as hypersensitivity; auto-immune diseases, such as multiple sclerosis; bone diseases; and certain neurodegenerative diseases, such as Alzheimer's and Parkinson's disease. Such inhibitors are also useful for the repopulation of hematopoietic cells following chemo- and radiation therapy and for prolonging organ viability for use in transplantation.

ICE/ced-3 inhibitors represent a class of compounds useful for the control of the above-listed disease states. Peptide and peptidyl inhibitors of ICE have been described. However, such inhibitors have been typically characterized by undesirable pharmacologic properties, such as poor oral absorption, poor stability and rapid metabolism. Plattner, J. J. and D. W. Norbeck, in Drug Discovery Technologies, C. R. Clark and W. H. Moos, Eds. (Ellis Horwood, Chichester, England, 1990), pp. 92-126. These undesirable properties have hampered their development into effective drugs.

Accordingly, the need exists for compounds that can effectively inhibit the action of the ICE/ced-3 family of proteases, for use as agents for preventing unwanted apoptosis and for treating chronic and acute forms of IL-1 mediated diseases, such as inflammatory, autoimmune or neurodegenerative diseases. The present invention satisfies this need and provides further related advantages.

### BRIEF SUMMARY OF THE INVENTION

In general, the compounds of this invention incorporate an aryl or heteroaryl substituted acyl group as a dipeptide mimetic. The resulting compounds exhibit improved properties relative to their peptidic counterparts, for example, such as improved cell penetration or improved absorption and metabolic stability resulting in enhanced bioavailability.

One aspect of the instant invention is the compounds of the Formula I:

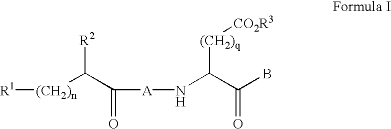

wherein A, B, n, q, R¹, R² and R³ are as defined below, as well as pharmacuetically acceptable salts thereof.

A further aspect of the instant invention is a pharmaceutical composition comprising a compound of the above Formula I and a pharmaceutically-acceptable carrier therefor.

Another aspect of this invention involves a method for treating an autoimmune disease comprising administering an effective amount of a pharmaceutical composition discussed above to a patient in need of such treatment.

Yet another aspect of the instant invention is a method for treating an inflammatory disease comprising administering an effective amount of a pharmaceutical composition discussed above to a patient in need of such treatment.

A further aspect of the instant invention is a method for treating a neurodegenerative disease comprising administering an effective amount of a pharmaceutical composition discussed above to a patient in need of such treatment.

Another aspect of the instant invention is a method of preventing ischemic injury to a patient suffering from a disease associated with ischemic injury comprising administering an effective amount of the pharmaceutical composition discussed above to a patient in need of such treatment.

A further aspect of the instant invention is a method for expanding of hematopoietic cell populations and/or enhancing their survival by contacting the cells with an effective amount of the pharmaceutical composition discussed above. Cell populations included in the method of the invention include (but are not limited to) granulocytes, monocytes, erthrocytes, lymphocytes and platelets for use in cell transfusions.

An alternate aspect of the instant invention is a method of prolonging the viability of an organ that has been removed from the donor for the purpose of a future transplantation procedure, which comprises applying an effective amount of the pharmaceutical composition discussed above to the organ, thereby prolonging the viability of the organ as compared to an untreated organ. The organ may be an intact organ, or isolated cells derived from an organ (e.g., isolated pancreatic islet cells, isolated dopaminergic neurons, blood or hematopoietic cells).

These and other aspects of this invention will be evident upon reference to the following detailed description.

### DETAILED DESCRIPTION OF THE INVENTION

As mentioned above, one aspect of the instant invention is the compounds of the Formula I:

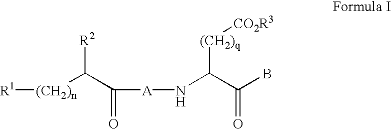

wherein:

n is 0, 1 or 2;

q is 1 or 2;

A is a natural or unnatural amino acid of Formula IIa-i:

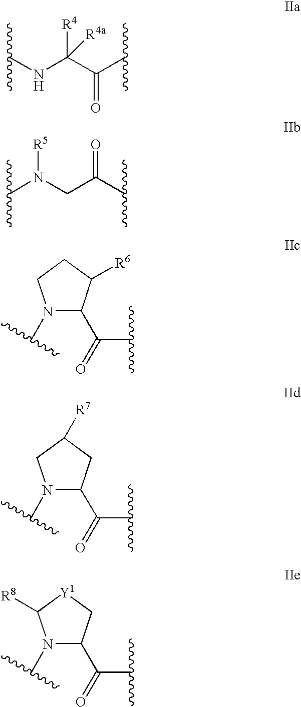

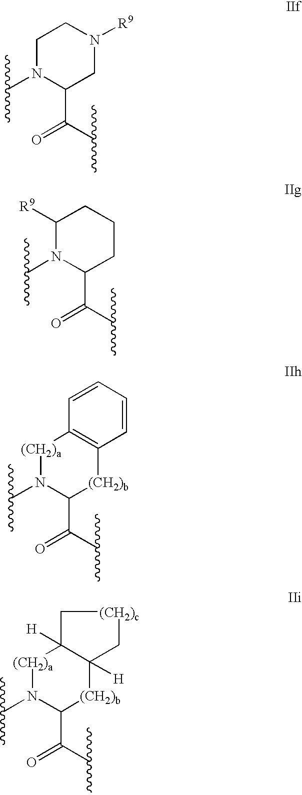

B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₘcycloalkyl, (CH₂)ₘphenyl, (CH₂)ₘ(substituted phenyl), (CH₂)ₘ(1 or 2-naphthyl), (CH₂)ₘheteroaryl, halomethyl, CO₂R¹³, CONR¹⁴R¹⁵, CH₂ZR¹⁶, CH₂OCO(aryl), CH₂OCO(substituted aryl), CH₂OCO(heteroaryl), CH₂OCO(substituted heteroaryl), or CH₂OPO(R¹⁷)R¹⁸, where Z is an oxygen or a sulfur atom, or B is a group of the Formula IIa-c:

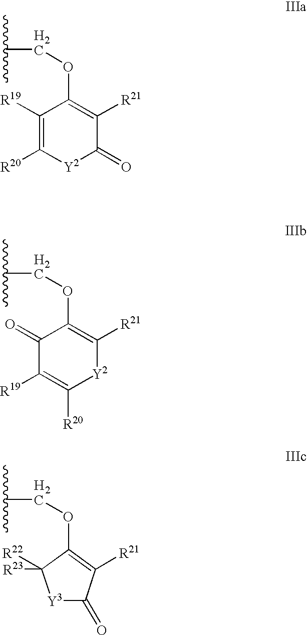

R¹ is cycloalkyl, substituted cycloalkyl, phenyl, substituted phenyl, naphthyl, substituted naphthyl, heteroaryl, and substituted heteroaryl;

R² is hydrogen, alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) substituted or unsubstituted pyridyl, thienyl, furyl, thiazolyl, imidazolyl, pyrazolyl, isoxazolyl, pyrazinyl, pyrimidyl, triazinyl, tetrazolyl, and indolyl;

R³ is hydrogen, alkyl, cycloalkyl, (cycloalkyl)alkyl, phenylalkyl, or substituted phenylalkyl;

and wherein

R⁴ is alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) pyridyl, thienyl, furyl, thiazolyl, imidazolyl, pyrazolyl, isoxazolyl, pyrazinyl, pyrimidyl, triazinyl, tetrazolyl, and indolyl;

R^{4a} is hydrogen or methyl, or R⁴ and R^{4a} taken together are —(CH₂)_{d}— where d is an interger from 2 to 6;

R⁵ is phenyl, substituted phenyl, (CH₂)ₚphenyl, (CH₂)ₚ(substituted phenyl), cycloalkyl, or benzofused cycloalkyl;

R⁶ is hydrogen, alkyl, cycloalkyl, phenyl, substituted phenyl, (CH₂)ₘcycloalkyl, (CH₂)ₘphenyl, (CH₂)ₘ(substituted phenyl), or (CH₂)ₘ(1 or 2-naphthyl);

R⁷ is hydrogen, fluorine, oxo (i.e., ═O), alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹¹, SR¹²,or NHCOR¹⁰;

R⁸ is hydrogen, oxo, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R⁹ is alkyl, cycloalkyl, (CH₂)ₘcycloalkyl, (CH₂)ₘphenyl, (CH₂)ₘ(substituted phenyl), (CH₂)ₘ(1 or 2-naphthyl), or COR¹⁰;

R¹⁰ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹³, or NR¹⁴R¹⁵;

R¹¹ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹² is alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹³ is alkyl, cycloalkyl, (CH₂)ₘcycloalkyl, (CH₂)ₘphenyl, (CH₂)ₘ(substituted phenyl), or (CH₂)ₘ(1 or 2-naphthyl);

R¹⁴ is hydrogen, alkyl, cycloalkyl, phenyl, substituted phenyl, naphthyl, substituted naphthyl, (CH₂)ₘcycloalkyl, (CH₂)ₘphenyl, (CH₂)ₘ(substituted phenyl), or (CH₂)ₘ(1 or 2-naphthyl);

R¹⁵ is hydrogen or alkyl; or

R¹⁴ and R¹⁵ taken together form a five, six or seven membered carbocyclic or heterocyclic ring, such as morpholine or N-substituted piperazine;

R¹⁶ is phenyl, substituted phenyl, naphthyl, substituted naphthyl, heteroaryl, (CH₂)ₘphenyl, (CH₂)ₘ(substituted phenyl), (CH₂)ₘ(1 or 2-naphthyl), or (CH₂)ₘheteroaryl;

R¹⁷ and R¹⁸ are independently alkyl, cycloalkyl, phenyl, substituted phenyl, naphthyl, or phenylalkyl, substituted phenylalkyl, or (cycloalkyl)alkyl;

R¹⁹ and R²⁰ are independently hydrogen, alkyl, phenyl, substituted phenyl, (CH₂)ₘphenyl, or (CH₂)ₘ(substituted phenyl), or R¹⁹ and R²⁰ taken together are —(CH═CH)₂—;

R²¹ is hydrogen, alkyl, phenyl, substituted phenyl, (CH₂)ₘphenyl, (CH₂)ₘ(substituted phenyl);

R²², R²³ and R²⁴ are independently hydrogen or alkyl;

Y¹ is CH₂, (CH₂)₂, (CH₂)₃, or S;

Y² is O or NR²⁴;

Y³ is CH₂, O, or NR²⁴;

a is 0 or 1 and b is 1 or 2, provided that when a is 1 then b is 1;

c is 1 or 2, provided that when c is 1 then a is 0 and b is 1;

m is 1, 2, 3 or 4; and

p is 1 or 2;

or a pharmaceutically acceptable salt thereof.

As used herein, the term “alkyl” means a straight or branched C₁ to C₈ carbon chain such as methyl, ethyl, tert-butyl, iso-propyl, n-octyl, and the like. The term “lower alkyl” means a straight or branched C₁ to C₆ carbon chain, such as methyl, ethyl, iso-propyl, and the like.

The term “cycloalkyl” means a mono-, bi-, or tricyclic ring that is either fully saturated or partially unsaturated. Examples of such a ring include cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted with one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more, and preferably one or two, substituents chosen from halogen, hydroxy, protected hydroxy, cyano, nitro, trifluoromethyl, alkyl, alkoxy, acyl, acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(lower alkyl)carboxamide, protected N-(lower alkyl)carboxamide, N,N-di(lower alkyl)carboxamide, N-((lower alkyl)sulfonyl)amino, N-(phenylsulfonyl)amino or by a substituted or unsubstituted phenyl group, such that in the latter case a biphenyl or naphthyl group results.

Examples of the term “substituted phenyl” includes a mono- or di(halo)phenyl group such as 2-, 3- or 4-chlorophenyl, 2,6-dichlorophenyl, 2,5-dichlorophenyl, 3,4-dichlorophenyl, 2-,3- or 4-bromophenyl, 3,4-dibromophenyl, 3-chloro-4-fluorophenyl, 2-, 3- or 4-fluorophenyl and the like; a mono or di(hydroxy)phenyl group such as 2-, 3-, or 4-hydroxyphenyl, 2,4-dihydroxyphenyl, the protected-hydroxy derivatives thereof and the like; a nitrophenyl group such as 2-, 3-, or 4-nitrophenyl; a cyanophenyl group, for example, 2-,3- or 4-cyanophenyl; a mono- or di(alkyl)phenyl group such as 2-, 3-, or 4-methylphenyl, 2,4-dimethylphenyl, 2-, 3- or 4-(iso-propyl)phenyl, 2-, 3-, or 4-ethylphenyl, 2-, 3- or 4-(n-propyl)phenyl and the like; a mono or di(alkoxy)phenyl group, for example, 2,6-dimethoxyphenyl, 2-, 3- or 4-(iso-propoxy)phenyl, 2-, 3- or 4-(t-butoxy)phenyl, 3-ethoxy-4-methoxyphenyl and the like; 2-, 3- or 4-trifluoromethylphenyl; a mono- or dicarboxyphenyl or (protected carboxy)phenyl group such as 2-, 3- or 4-carboxyphenyl or 2,4-di(protected carboxy)phenyl; a mono- or di(hydroxymethyl)phenyl or (protected hydroxymethyl)phenyl such as 2-, 3- or 4-(protected hydroxymethyl)phenyl or 3,4-di(hydroxymethyl)phenyl; a mono- or di(aminomethyl)phenyl or (protected aminomethyl)phenyl such as 2-, 3- or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like.

The term “phenylalkyl” means one of the above phenyl groups attached to one of the above-described alkyl groups, and the term “substituted phenylalkyl means that either the phenyl or the alkyl, or both, are substituted with one or more of the above-identified substituents. Examples of such groups include 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl, and the like.

The term “substituted naphthyl” means a naphthyl group substituted with one or more of the above-identified substituents, and the term “(1 or 2 naphyl)alkyl” means a naphthyl attached to one of the above-described alkyl groups at the 1 or 2 position.

The terms “halo” and “halogen” refer to the fluoro, chloro, bromo or iodo groups. These terms may also be used to describe one or more halogens, which are the same or different. Preferred halogens in the context of this invention are chloro and fluoro.

The term “aryl” refers to aromatic five and six membered carbocyclic rings. Six membered rings are preferred.

The term “heteroaryl” denotes optionally substituted aromatic five-membered or six-membered heterocyclic rings that have 1 to 4 heteroatoms, such as oxygen, sulfur and/or nitrogen atoms, in particular nitrogen, either alone or in conjunction with sulfur or oxygen ring atoms.

The following ring systems are representative examples of the heterocyclic radicals denoted by the term “heteroaryl” (whether substitued or unsubstituted): thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzothiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, lower alkyl, substituted alkyl, cycloalkyl, substituted cycloalkyl, (cycloalkyl)alkyl, substituted (cycloalkyl)alkyl, phenyl, substituted phenyl, phenylalkyl, and substituted phenylalkyl groups.

Substituents for the heteroaryl group are as defined above, or as set forth below. As used in conjunction with the above substituents for heteroaryl rings, “trihalomethyl” can be trifluoromethyl, trichloromethyl, tribromomethyl or triiodomethyl, “lower alkoxy” means a C₁ to C₄ alkoxy group, similarly, “lower alkylthio” means a C₁ to C₄ alkylthio group. The term “substituted lower alkyl” means the above-defined lower alkyl group substituted from one to three times by a hydroxy, protected hydroxy, amino, protected amino, cyano, halo, trifluoromethyl, mono-substituted amino, di-substituted amino, lower alkoxy, lower alkylthio, carboxy, protected carboxy, or a carboxy, amino, and/or hydroxy salt.

As used in conjunction with the substituents for the heteroaryl rings, the terms “substituted (cycloalkyl)alkyl” and “substituted cycloalkyl” are as defined above substituted with the same groups as listed for a “substituted alkyl” group. The term “(monosubstituted)amino” refers to an amino group with one substituent chosen from the group consisting of phenyl, substituted phenyl, alkyl, substituted alkyl, C₁ to C₇ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted)amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different. The term “heteroaryl(alkyl)” denotes an alkyl group as defined above, substituted at any position by a heteroaryl group, as above defined.

Furthermore, the above optionally substituted five-membered or six-membered heterocyclic rings can optionally be fused to a aromatic 5-membered or 6-membered aryl or heteroaryl ring system. For example, the rings can be optionally fused to an aromatic 5-membered or 6-membered ring system such as a pyridine or a triazole system, and preferably to a benzene ring.

The term “pharmaceutically-acceptable salt” encompasses those salts that form with the carboxylate anions and includes salts formed with the organic and inorganic cations such as those chosen from the alkali and alkaline earth metals (for example, lithium, sodium, potassium, magnesium, barium and calcium); and ammonium ion; and the organic cations (for example, dibenzylammonium, benzylammonium, 2-hydroxyethylammonium, bis(2-hydroxyethyl)ammonium, phenylethylbenzylammonium, dibenzylethylenediammonium, and like cations). Other cations encompassed by the above term include the protonated form of procaine, quinine and N-methylglucosamine, the protonated forms of basic amino acids such as glycine, ornithine, histidine, phenylglycine, lysine, and arginine. Furthermore, any zwitterionic form of the instant compounds formed by a carboxylic acid and an amino group is referred to by this term. A preferred cation for the carboxylate anion is the sodium cation. Furthermore, the term includes salts that form by standard acid-base reactions with basic groups (such as amino groups) and includes organic or inorganic acids. Such acids include hydrochloric, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and the like acids.

The compounds of Formula I may also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, β-(trimethylsilyl)ethyl, β-(di(n-butyl)methylsilyl)ethyl, p-toluenesulfonylethyl, 4-nitrobenzylsulfonylethyl, allyl, cinnamyl, 1-(trimethylsilylmethyl)-propenyl and like moieties. The species of carboxy-protecting group employed is not critical so long as the derivatized carboxylic acid is stable to the conditions of subsequent reaction(s) and can be removed at the appropriate point without disrupting the remainder of the molecule. Further examples of these groups are found in C. B. Reese and E. Haslam, “Protective Groups in Organic Chemistry,” J. G. W. McOmie, Ed., Plenum Press, New York, N.Y., 1973, Chapter 5, respectively, and T. W. Greene and P. G. M. Wuts, “Protective Groups in Organic Synthesis,” 2nd ed., John Wiley and Sons, New York, N.Y., 1991, Chapter 5, each of which is incorporated herein by reference. A related term is “protected carboxy,” which refers to a carboxy group substituted with one of the above carboxy-protecting groups.

The term “hydroxy-protecting group” refers to readily cleavable groups bonded to hydroxyl groups, such as the tetrahydropyranyl, 2-methoxyprop-2-yl, 1-ethoxyeth-1-yl, methoxymethyl, β-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, 2,2,2-trichloroethoxycarbonyl, and the like.

Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” Second Edition, John Wiley and Sons, New York, N.Y., 1991, Chapters 2 and 3. A preferred hydroxy-protecting group is the tert-butyl group. The related term “protected hydroxy” denotes a hydroxy group bonded to one of the above hydroxy-protecting groups.

The term “amino-protecting group” as used herein refers to substituents of the amino group commonly employed to block or protect the amino functionality while reacting other functional groups of the molecule. The term “protected (monosubstituted)amino” means there is an amino-protecting group on the monosubstituted amino nitrogen atom.

Examples of such amino-protecting groups include the formyl (“For”) group, the trityl group, the phthalimido group, the trichloroacetyl group, the trifluoroacetyl group, the chloroacetyl, bromoacetyl, and iodoacetyl groups, urethane-type protecting groups, such as t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl(“Ddz”), 2-(p-toluyl)propyl-2-oxycarbonyl, cyclopentanyloxycarbonyl, 1-methylcyclopentanyl-oxycarbonyl, cyclohexanyloxy-carbonyl, 1-methyl-cyclohexanyloxycarbonyl, 2-methylcyclohexanyl-oxycarbonyl, 2-(4-toluylsulfonyl)ethoxycarbonyl, 2-(methylsulfonyl)ethoxycarbonyl, 2-(triphenylphosphino)-ethoxycarbonyl, 9-fluorenylmethoxycarbonyl(“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloroethoxycarbonyl, 2-ethynyl-2-propoxycarbonyl, cyclopropylmethoxycarbonyl, isobornyloxycarbonyl, 1-piperidyloxycarbonyl, benzyloxycarbonyl(“Cbz”), 4-phenylbenzyloxycarbonyl, 2-methylbenzyloxycarbonyl, α-2,4,5,-tetramethylbenzyl-oxycarbonyl(“Tmz”), 4-methoxybenzyloxycarbonyl, 4-fluorobenzyloxycarbonyl, 4-chlorobenzyloxycarbonyl, 3 -chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 4-(decyloxy)benzyloxycarbonyl and the like; the benzoylmethylsulfonyl group, the 2,2,5,7,8-pentamethylchroman-6-sulfonyl group (“PMC”), the dithiasuccinoyl(“Dts”) group, the 2-(nitro)phenyl-sulfenyl group (“Nps”), the diphenylphosphine oxide group, and like amino-protecting groups. The species of amino-protecting group employed is not critical so long as the derivatized amino group is stable to the conditions of the subsequent reaction(s) and can be removed at the appropriate point without disrupting the remainder of the molecule. Preferred amino-protecting groups are Boc, Cbz and Fmoc. Further examples of amino-protecting groups embraced by the above term are well known in organic synthesis and the peptide art and are described by, for example, T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised Ed., Springer-Verlag, New York, N.Y., 1984 and 1993, and J. M. Stewart and J. D. Young, “Solid Phase Peptide Synthesis,” 2nd Ed., Pierce Chemical Co., Rockford, Ill., 1984, E. Atherton and R. C. Shephard, “Solid Phase Peptide Synthesis—A Practical Approach” IRL Press, Oxford, England (1989), each of which is incorporated herein by reference. The related term “protected amino” defines an amino group substituted with an amino-protecting group discussed above.

The terms “natural and unnatural amino acid” refers to both the naturally occurring amino acids and other non-proteinogenic α-amino acids commonly utilized by those in the peptide chemistry arts when preparing synthetic analogues of naturally occurring peptides, including D and L forms. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, omithine and lysine. Examples of unnatural alpha-amino acids include hydroxylysine, citrulline, kynurenine, (4-aminophenyl)alanine, 3-(2′-naphthyl)alanine, 3-(1′-naphthyl)alanine, methionine sulfone, (t-butyl)alanine, (t-butyl)glycine, 4-hydroxyphenyl-glycine, aminoalanine, phenylglycine, vinylalanine, propargyl-gylcine, 1,2,4-triazolo-3-alanine, thyronine, 6-hydroxytryptophan, 5-hydroxytryptophan, 3-hydroxy-kynurenine, 3-aminotyrosine, trifluoromethylalanine, 2-thienylalanine, (2-(4-pyridyl)ethyl)cysteine, 3,4-dimethoxy-phenylalanine, 3-(2′-thiazolyl)alanine, ibotenic acid, 1-amino-1-cyclopentane-carboxylic acid, 1-amino-1-cyclohexanecarboxylic acid, quisqualic acid, 3-(trifluoromethylphenyl)alanine, (cyclohexyl)glycine, thiohistidine, 3-methoxytyrosine, norleucine, norvaline, alloisoleucine, homoarginine, thioproline, dehydro-proline, hydroxyproline, homoproline, indoline-2-carboxylic acid, 1,2,3,4-tetrahydroisoquinoline-3-carboxylic acid, 1,2,3,4-tetrahydroquinoline-2-carboxylic acid, α-amino-n-butyric acid, cyclohexylalanine, 2-amino-3-phenylbutyric acid, phenylalanine substituted at the ortho, meta, or para position of the phenyl moiety with one or two of the following groups: a (C₁ to C₄)alkyl, a (C₁ to C₄)alkoxy, a halogen or a nitro group, or substituted once with a methylenedioxy group; β-2- and 3-thienylalanine; β-2- and 3-furanylalanine; β-2-, 3- and 4-pyridylalanine; β-(benzothienyl-2- and 3-yl)alanine; β-(1- and 2-naphthyl)alanine; O-alkylated derivatives of serine, threonine or tyrosine; S-alkylated cysteine, S-alkylated homocysteine, the O-sulfate, O-phosphate and O-carboxylate esters of tyrosine; 3-(sulfo)tyrosine, 3-(carboxy)tyrosine, 3-(phospho)tyrosine, the 4-methane-sulfonic acid ester of tyrosine, 4-methanephosphonic acid ester of tyrosine, 3,5-diiodotyrosine, 3-nitrotyrosine, ε-alkyllysine, and delta-alkyl omithine. Any of these α-amino acids may be substituted with a methyl group at the alpha position, a halogen at any position of the aromatic residue on the a-amino side chain, or an appropriate protective group at the O, N, or S atoms of the side chain residues. Appropriate protective groups are discussed above.

Depending on the choice of solvent and other conditions known to the practitioner skilled in the art, compounds of this invention may also take the ketal or acetal form, which forms are included in the instant invention. In particular, when R³ is hydrogen compounds of compounds of Formula Ia may exist in the cyclic ketal or acetal form Formula Ia′ shown below:

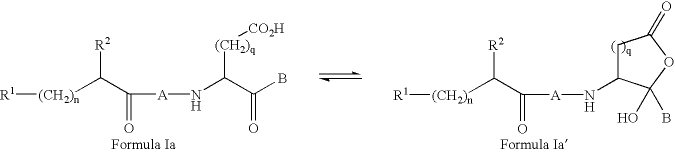

Similarly, when R³ of Formula I is a moiety other than hydrogen, and depending upon the choice of solvents as noted above (e.g., R³OH), the compounds of the cyclic ketal or acetal form include compounds having Formula Ia″ as shown below:

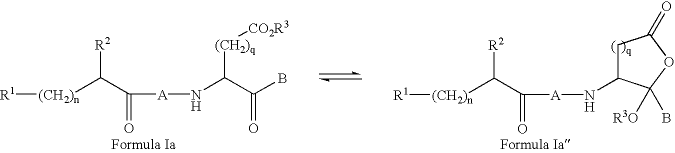

In addition, it should be understood that the equilibrium forms of the compounds of this invention may include tautomeric forms. All such forms of these compounds are expressly included in the present invention.

The compounds of this invention may be modified by appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of exertion. In addition, the compounds may be altered to pro-drug form such that the desired compound is created in the body of the patient as the result of the action of metabolic or other biochemical processes on the pro-drug. Some examples of pro-drug forms include ketal, acetal, oxime, and hydrazone forms of compounds which contain ketone or aldehyde groups, especially where they occur in the group donated as “A” in Formula I or the modified aspartic acid residue attached to the group denoted as “A”.

Compounds of this invention with respect to the groups R¹ and R² in Formula I, include those wherein:

R¹ is cycloalkyl (such as cyclohexyl), substituted phenyl (such as 2-substituted phenyl), naphthyl, or substituted naphthyl;

R² is hydrogen, lower alkyl, (CH₂)ₚCO₂R³, (CH₂)ₘphenyl, (CH₂)ₘ(substituted phenyl), (CH₂)ₘ(1 or 2-naphthyl), or (CH₂)ₘtetrazolyl, where p is 1 or 2,m is 1 or 2;

R³ is hydrogen or alkyl;

q is 1; and

n is 0 or 1.

Other compounds of this invention with respect to the groups R¹ and R² in Formula I, include those wherein:

R¹ is cyclohexyl, substituted phenyl, naphthyl, or substituted naphthyl; and

R² is (CH₂)ₘtetrazolyl, where m is 1 or 2; and

Compounds of this invention with respect to the group “A” in Formula I, include those of Formula Ia wherein:

R⁴ is lower alkyl, cycloalkyl, phenyl, substituted phenyl, (CH₂)ₘNH₂, (CH₂)ₚOR¹¹, (CH₂)ₚSR¹², (CH₂)ₘcycloalkyl, (CH₂)ₘphenyl, (CH₂)ₘ(substituted phenyl), or (CH₂)ₘ(1 or 2-naphthyl);

R¹¹ is hydrogen, lower alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹² is lower alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl); and

m is 1, 2, 3, 4and p is 1 or 2.

Compounds of this invention with respect to the group “A” in Formula I, also include those of Formula IIb wherein:

R⁵ is phenyl, substituted phenyl, (CH₂)ₚphenyl, (CH₂)ₚ(substituted phenyl), cycloalkyl, or 2-indanyl; and

p is 1 or 2.

Another group of compounds with respect to the group “A” in Formula I, include those of Formula IId wherein:

R⁷ is hydrogen, fluorine, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹¹, or SR¹²;

R¹¹ and R¹² are independently cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl); and

m is 1, 2, 3 or 4.

A forth group of compounds with respect to the group “A” in Formula I, include those of Formula IIe wherein:

R⁸ is hydrogen, oxo, cycloalkyl, phenyl, substituted phenyl, or naphthyl; and

Y¹ is CH₂, (CH₂)₂, (CH₂)₃, or S.

Another group of compounds with respect to the group “A” in Formula I, include those of Formula IIh wherein:

a is 0and b is 1 or 2.

Compounds of this invention with respect to the group “B” in Formula I, include those wherein:

B is hydrogen, 2-benzoxazolyl, substituted 2-oxazolyl, CH₂ZR¹⁶, CH₂OCO(aryl), or CH₂OPO(R¹⁷)R¹⁸, where Z is O or S;

R¹⁶ is phenyl, substituted phenyl, naphthyl, substituted naphthyl, heteroaryl, (CH₂)ₘphenyl, (CH₂)ₘ(substituted phenyl), (CH₂)ₘ(1 or 2-naphthyl), or (CH₂)ₘheteroaryl; and

R¹⁷ and R¹⁸ are independently alkyl, cycloalkyl, phenyl, substituted phenyl, naphthyl, phenylalkyl, substituted phenylalkyl and (cycloalkyl)alkyl.

Another group of compounds with respect to the group “B” in Formula I, include those of Formula IIIa-c wherein:

Y² is O or NR²⁴;

Y³ is CH₂, O, or NR²⁴;

R¹⁹ and R²⁰ are independently hydrogen, alkyl, phenyl, or R¹⁹ and R²⁰ taken together are —(CH═CH)₂—;

R²¹ is hydrogen, alkyl, phenyl, substituted phenyl, (CH₂)ₘphenyl, or (CH₂)ₘ(substituted phenyl); and

R²², R²³ and R²⁴ are independently hydrogen or alkyl.

The compounds of Formula I may be synthesized using conventional techniques as discussed below. Advantageously, these compounds are conveniently synthesized from readily available starting materials. To this end, in the following synthetic schemes, q is 1, and corresponding compounds wherein q is 2 may be made in the same manner by employing the corresponding ethylene (—CH₂CH₂—) starting material in place of the methylene (—CH₂—) moiety.

One synthetic route for synthesizing the instant compounds is set forth in the following Scheme 1:

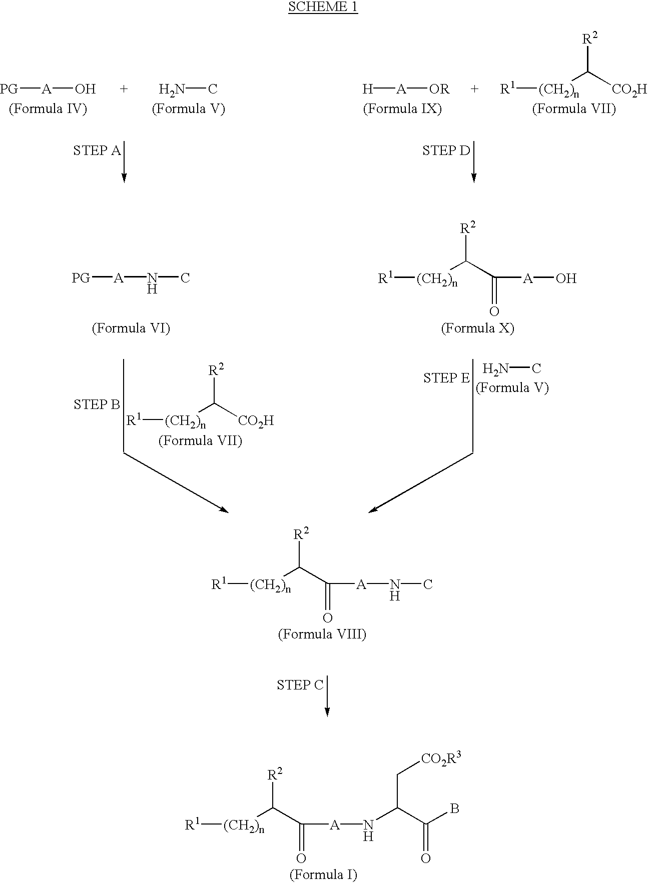

In the above Scheme 1, Formula (V), that is H₂N—C, is a modified aspartic acid residue of Formulas Va through Vd:

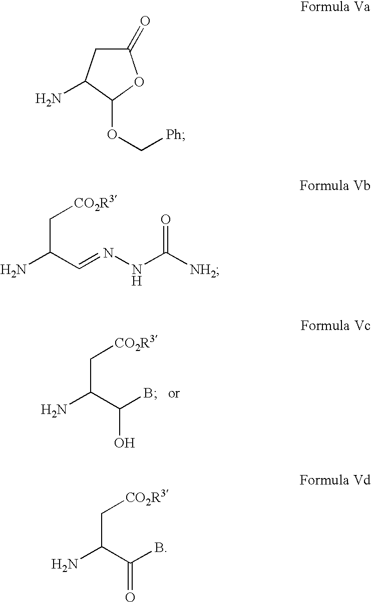

In the above Scheme 1, “PG” stands for an amino protecting group and “A” stands for a natural or unnatural amino acid of formula Ia through IIi, as discussed above. In Formula Vb through Vd, R^{3′} is a carboxyl protecting group as described in the definition of R³ in Formula I with the exception that R^{3′} cannot be a hydrogen atom.

The modified aspartic acids of Formula Va-d can be prepared by methods well known in the art. See, for example, European Patent Application 519,748; PCT Patent Application No. PCT/EP92/02472; PCT Patent Application No. PCT/US91/06595; PCT Patent Application No. PCT/US91/02339; European Patent Application No. 623,592; World Patent Application No. WO 93/09135; PCT Patent Application No. PCT/US94/08868; European Patent Application No. 623,606; European Patent Application No. 618,223; European Patent Application No. 533,226; European Patent Application No. 528,487; European Patent Application No. 618,233; PCT Patent Application No. PCT/EP92/02472; World Patent Application No. WO 93/09135; PCT Patent Application No. PCT/US93/03589; and PCT Patent Application No. PCT/US93/00481, all of which are herein incorporated by reference.

The coupling reactions carried out under Step A are performed in the presence of a standard peptide coupling agent such as the combination of the combination of dicyclohexylcarbodiimide(DCC) and 1-hydroxy-benzotriazole(HOBt), as well as the BOP (benzotriazolyloxy-tris-(dimethylamino)phosphonium hexafluorophosphate) reagent, pyBOP (benzotriazolyloxy-tris(N-pyrolidinyl)phosphoniumhexafluorophosphate), HBTU (O-benzotriazolyly-tetramethylisouronium-hexafluorophosphate), and EEDQ (1-ethyloxycarbonyl-2-ethyloxy-1,2-dihydroquinoline)reagents, the combination of 1-ethyl(3,3′-dimethyl-1′-aminopropyl)carbodiimide (EDAC) and HOBt, and the like, as discussed in J. Jones, “Amino Acid and Peptide Synthesis,” Steven G. Davis ed., Oxford University Press, Oxford, pp. 25-41 (1992); M. Bodanzky, “Principles of Peptide Synthesis,” Hafner et al. ed., Springer-Verlag, Berlin Heidelberg, pp. 9-52 and pp. 202-251 (1984); M. Bodanzky, “Peptide Chemistry, A Practical Textbook,” Springer-Verlag, Berlin Heidelberg, pp. 55-73 and pp. 129-180; and Stewart and Young, “Solid Phase Peptide Synthesis,” Pierce Chemical Company, (1984), all of which are herein incorporated by reference. The amino protecting group is then removed and the resulting amine is coupled to the (substituted) carboxylic acid of Formula VII (Step B). Again, this coupling reaction uses the standard peptide coupling reactions mentioned above.

Alternatively, the substituted carboxylic acid of Formula VII can be coupled to an amino ester of Formula IX (Step D). Again, this coupling reaction uses the standard peptide coupling reactions mentioned above. In Formula IX, the group R is a carboxyl protecting group such as methyl, allyl, benzyl or tert-butyl. After removal of the carboxyl protecting group under standard conditions well known in the art, the resulting carboxylic acid is coupled to amine V using the standard peptide coupling methods described above (Step E).

In the case where the coupling reaction depicted by either Step A or Step E was carried out with the amino alcohol of Formula Vc, the alcohol moiety must be oxidized to the corresponding carbonyl compound prior to removal of the protecting groups. Preferred methods for the oxidation reaction include Swern oxidation (oxalyl chloride-dimethyl sulfoxide, methylene chloride at −78° C. followed by triethylamine); and Dess-Martin oxidation (Dess-Martin periodinane, t-butanol, and methylene chloride.) The protecting groups contained in substructures of the Formula Va-d, VII and A are removed by methods well known in the art. These reactions and removal of some or all of the protecting groups are involved in Step C in the above Scheme 1.

An alternative synthetic route for synthesizing the instant compounds is set forth in the following Scheme 2:

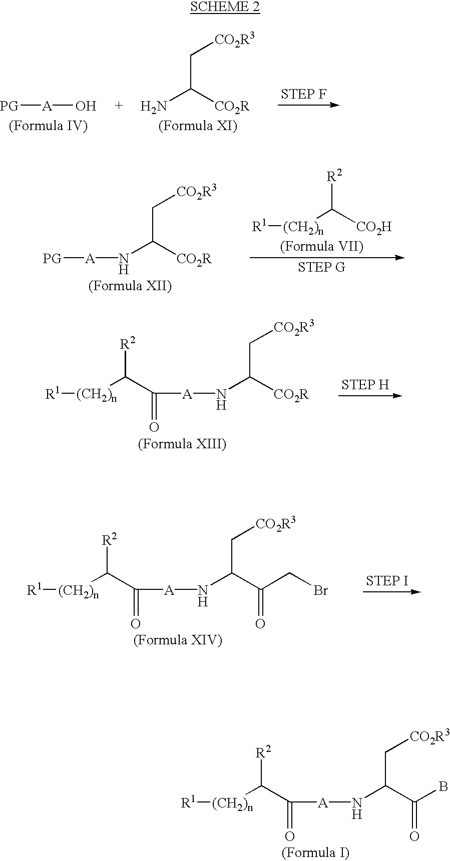

In the above Scheme 2, “PG” stands for an amino protecting group and “A” stands for a natural or unnatural amino acid of formula Ia through IIi, as discussed above. The group R is a carboxyl protecting group such as trimethylsilyl, methyl, allyl, benzyl or tert-butyl.

The coupling reactions carried out under Step F and Step G are performed in the presence of a standard peptide coupling agent as discussed above. In Step G, the amino protecting group must be removed prior to the coupling step. In Step H the alpha-carboxy protecting group R of the compound of Formula XIII is selectively removed and the resulting mono-carboxylic acid treated sequentially with diazomethane and hydrobromic acid to give the alpha-bromoketone of Formula XIV.

In Step I, the bromoketone of Formula XIV is treated with either R¹⁶Z-H, (aryl)-CO₂H, (heteroaryl)-CO₂H, or R¹⁷(R¹⁸)PO₂H in the presence of an inorganic base such as potassium carbonate or potassium fluoride in an inert solvent such as dimethyl formamide to give the corresponding compound of Formula I in which B is CH₂ZR¹⁶, CH₂OCO(aryl), CH₂OCO(heteroaryl), or CH₂OPO(R¹⁷)R¹⁸, respectively. Compounds of Formula I in which B is a fragment of Formula III may also be prepared in a similar fashion. The protecting groups contained in substructures of the Formula VII, XI and A are removed by methods well known in the art. These reactions and removal of some or all of the protecting groups are involved in Step I in the above Scheme 2.

An alternative method for the prepartion of compounds of the instant invention of Formula I in which R³ and B are both hydrogen (i.e., Formula Ib) is set forth below in Scheme 3:

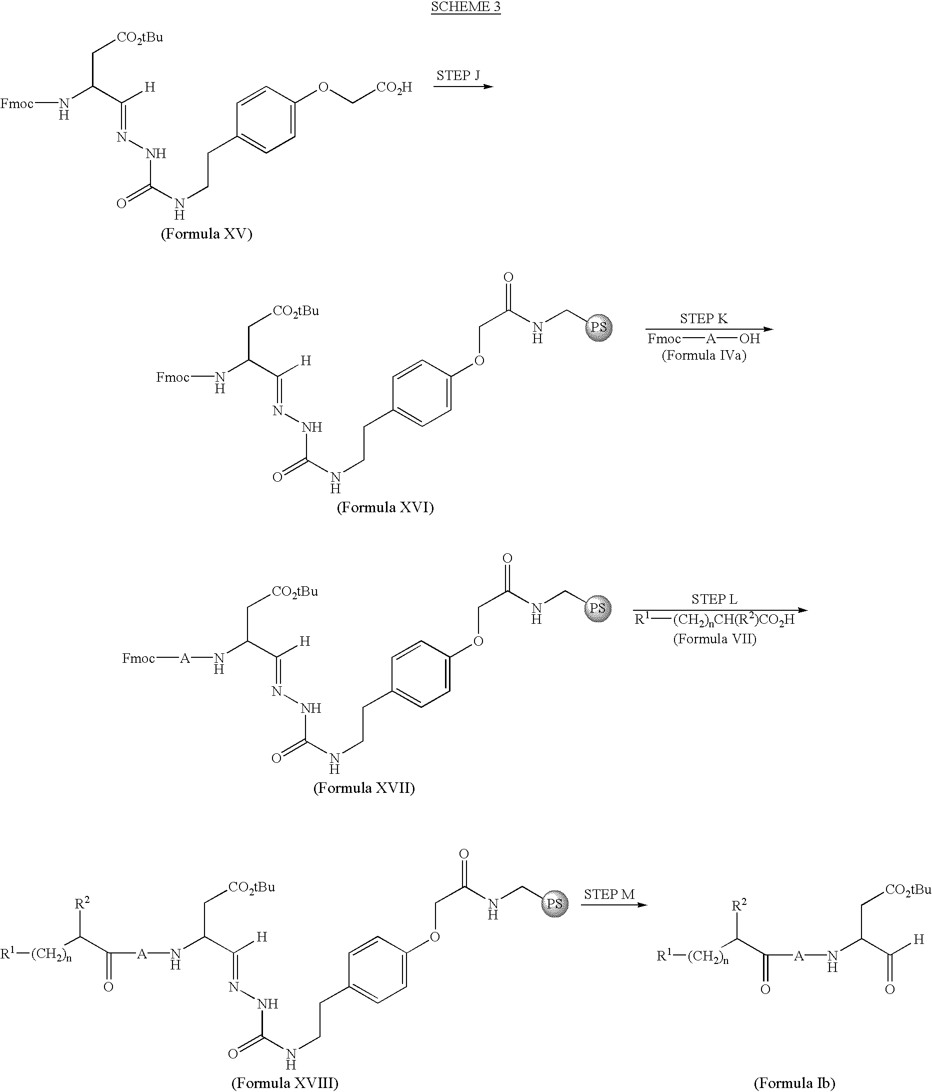

In Scheme 3, Fmoc is the amino protecting group 9-fluorenylmethoxycarbonyl and the shaded circle labeled “PS” represents polystryene resin.

The coupling of the acid of Formula XV to a primary amine on solid support, preferably aminomethyl polystyrene, is carried out using standard peptide coupling agents, preferably using benzotriazolyloxy-tris(N-pyrolidinyl)phosphoniumhexafluorophosphate (pyBOP) in a inert solvent such as dimethylformamide or N-methyl pyrrolidone (Step J). After removal of the Fmoc protecting group of XVI by treatment with pyrrolidine-dimethylformamide, the resulting amine is coupled to Fmoc-amino acid of Formula IVa using standard peptide coupling conditions as discussed above (Step K).

In Step L the Fmoc protecting group of the compound of Formula XVII is removed again by treatment with with pyrrolidine-dimethylformamide and the resulting amine coupled to the (substituted)carboxylic acid of Formula VII again using standard peptide coupling conditions as discussed above. The tert-butyl ester of the compound of Formula XVIII is removed by treatment with trfluoroacetic acid-methylene chloride in the presence of a trapping agent such as anisole and the resulting acid cleaved from the solid support by treatment with 37% aqueous formaldehyde/acetic acid/tetrahydrofuran/trifluoroacetic acid, preferably in a ratio of 1/1/5/0.025, to give the aspartyl aldehyde of Formula Ib (Step M).

Pharmaceutical compositions of this invention comprise any of the compounds of the present invention, and pharmaceutically acceptable salts thereof, with any pharmaceutically acceptable carrier, adjuvant or vehicle (hereinafter collectively referred to as “pharmaceutically-acceptable carriers”). Pharmaceutically acceptable carriers, adjuvants and vehicles that may be used in the pharmaceutical compositions of this invention include, but are not limited to, ion exchange, alumina, aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene-polyoxypropylene-block polymers, polyethylene glycol and wool fat, and the like.

The pharmaceutical compositions of this invention may be administered orally, parenterally, by inhalation spray, topically, rectally, nasally, buccally, vaginally or by an implanted reservoir. Oral and parenteral administration are preferred. The term “parenteral” as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to, capsules, tablets, and aqueous suspensions and solutions. In the case of tablets for oral use, carriers that are commonly used include lactose and cornstarch. Lubricating agents, such as magnesium stearate, are also typically added. For oral administration in capsule form useful diluents include lactose and dried cornstarch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature. Such materials include, but are not limited to, cocoa butter, beeswax and polyethylene glycols.

Topical administration of the pharmaceutical compositions of this invention is especially useful when the desired treatment involves areas or organs readily accessible to topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene, polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-applied transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The compounds of this invention may be used in combination with either conventional anti-inflammatory agents or with matrix metalloprotease inhibitors, lipoxygenase inhibitors and antagonists of cytokines other than IL-1β.

The compounds of this invention can also be administered in combination with immunomodulators (e.g., bropirimine, anti-human alpha interferon antibody, IL-2, GM-CSF, methionine enkephalin, interferon alpha, diethyldithiocarbamate, tumor necrosis factor, naltrexons and rEPO) or with prostaglandins, to prevent or combat IL-1-mediated disease symptoms such as inflammation.

When the compounds of this invention are administered in combination therapies with other agents, they may be administered sequentially or concurrently to the patient. Alternatively, pharmaceutical compositions according to this invention may be comprised of a combination of a compound of Formula I and another therapeutic or prophylactic agent mentioned above.

The disease states which may be treated or prevented by the instant pharmaceutical compositions include, but are not limited to, inflammatory diseases, autoimmune diseases and neurodegenerative diseases, and for inhibiting unwanted apoptosis involved in ischemic injury, such as ischemic injury to the heart (e.g., myocardial infarction), brain (e.g., stroke), and kidney (e.g., ischemic kidney disease). As a consequence of their ability to inhibit apoptosis, the present pharmaceutical compositions are also useful for the repopulation of hematopoietic cells of a patient following chemotherapy. Methods of administering an effective amount of the above-described pharmaceutical compositions to mammals, also referred to herein as patients, in need of such treatment (that is, those suffering from inflammatory diseases, autoimmune diseases, neurodegenerative diseases and for the repopulation of hematopoietic cells in cancer patients who have undergone chemotherapy) are another aspect of the instant invention. Finally, as a further consequence of their ability to inhibit apoptosis, the instant pharmaceutical compositions may be used in a method to prolong the viability of organs to be used in transplantations.

Inflammatory diseases that may be treated or prevented include, for example, septic shock, septicemia, and adult respiratory distress syndrome. Target autoimmune diseases include, for example, rheumatoid, arthritis, systemic lupus erythematosus, scleroderma, chronic thyroiditis, Graves' disease, autoimmune gastritis, insulin-dependent diabetes mellitus, autoimmune hemolytic anemia, autoimmune neutropenia, thrombocytopenia, chronic active hepatitis, myasthenia gravis and multiple sclerosis. Target neurodegenerative diseases include, for example, amyotrophic lateral sclerosis, Alzheimer's disease, Parkinson's disease, and primary lateral sclerosis. The pharmaceutical compositions of this invention may also be used to promote wound healing. Target diseases associated with harmful, apoptosis, in other words, those associated with ischemic injury, includes myocardial infarction, stroke, and ischemic kidney disease. The pharmaceutical compositions of this invention may also be used to treat infectious diseases, especially those involved with viral infections.

The term “effective amount” refers to dosage levels of the order of from about 0.05 milligrams to about 140 milligrams per kilogram of body weight per day for use in the treatment of the above-indicated conditions (typically about 2.5 milligrams to about 7 grams per patient per day). For example, inflammation may be effectively treated by the administration of from about 0.01 to 50 milligrams of the compound per kilogram of body weight per day (about 0.5 milligrams to about 3.5 grams per patient per day).

The amount of the compounds of Formula I that may be combined with the carrier materials to produce a single dosage form will vary depending upon the host treated and the particular mode of administration. For example, a formulation intended for the oral administration of humans may contain from 0.5 milligrams to 5 grams of a compound of Formula I combined with an appropriate and convenient amount of a pharmaceutically-acceptable carrier which may vary from about 5 to about 95 percent of the total composition. Dosage unit forms will generally contain between from about I milligram to about 500 milligrams of an active compound of Formula I.

It will be understood, however, that the specific “effective amount” for any particular patient will depend upon a variety of factors including the activity of the specific compound employed, the age, body weight, general health, sex, diet, time of administration, route of administration, rate of excretion, drug combination and the severity of the particular disease undergoing prevention or therapy.

Although this invention focuses on the use of the compounds disclosed herein for preventing and treating IL-1-mediated diseases, the compounds of this invention can also be used as inhibitory agents for other cysteine proteases.

The compounds of this invention are also useful as commercial reagents which effectively bind to the ICE/ced-3 family of cysteine protease or other cysteine proteases. As commercial reagents, the compounds of this invention, and their derivatives, may be used to block proteolysis of a target peptide or may be derivatized to bind to a stable resin as a tethered substrate for affinity chromatography applications. These and other uses which characterize commercial cystine protease inhibitors will be evident to those of ordinary skill in the art.

In order that this invention be more fully understood, the following examples are set forth. These examples are for the purpose of illustration only and are not to be construed as limiting the scope of the invention in any way.

In the following Examples, proton NMR spectra were obtained at 300 MHz; chemical shifts are quoted downfield from internal tetramethylsilane.

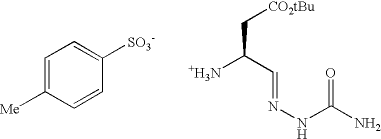

Preparation of (3S)-Amino-4-Oxobutanoic Acid tert-Butyl Ester Semicarbazone, p-Toluenesulfonate Salt

Part A: N-(Benzyloxycarbonyl)-L-(N-Methyl-N′-Methoxy aspartanide β-(tert-Butyl)Ester

To a solution of N-(benzyloxycarbonyl)-L-aspartic acid-β-(tert-butyl) ester (14.65 g, 45.3 mmol, Bachem) in CH₂Cl₂ (150 mL) at 0° C. (ice bath) under a nitrogen atmosphere was added 1-hydroxybenzotriazole hydrate (7.29 g, 47.6 mmol, Aldrich) followed by 1-ethyl-3-(3′,3′-dimethyl-1′-aminopropyl)carbodiimide hydrochloride (9.55 g, 49.8 mmol, Sigma). After stirring at 0° C. for 15 min., N,O-dimethylhydroxylamine hydrochloride (5.10 g, 52.3 mmol, Aldrich) and N-methylmorpholine (5.8 mL, 53 mmol, Aldrich) were added. The mixture was allowed to warm to room temperature over 3 hours then stirred at room temperature for 16 hours. The solution was concentrated under vacuum and the residue partitioned between ethyl acetate-5% KHSO₄ (200 mL each). The organic phase was washed in turn with 5% KHSO₄, saturated sodium bicarbonate and saturated sodium chloride solutions; dried over anhydrous sodium sulfate and evaporated to an oil. The oil was crystallized from hexane to give the title product (16.10 g, 97% yield) as a fluffy white crystalline solid. TLC (ethyl acetate), single spot (UV and PMA): Rf=0.37.

A similar procedure to the one above, starting with 29.3 g of N-(benzyloxycarbonyl)-L-aspartic acid-β-(tert-butyl)ester (2-fold scale up) gave 31.18 g (94% yield) of the title product.

Part B: (3S)-(Benzyloxycarbonyl)Amino-4-Oxobutanoic Acid tert-Butyl Ester Semicarbazone

To a solution of N-(benzyloxycarbonyl)-L-(N′-methyl-N′-methoxy)aspartamide-β-(tert-butyl)ester (15.50 g, 42.3 mmol) in anhydrous ether (400 mL) at 0° C. (ice bath) under a nitrogen atmosphere was added dropwise to a 1.0 M solution of LiAIH₄ in ether (22.0 mL, 22.0 mmol, Aldrich) at such a rate as to keep the reaction solution temperature between 0-5° C. (addition time 15-20 min). After the addition of the lithium aluminum hydride reagent was complete, the mixture was stirred at 0-5° C. for 1 hrs, then quenched by the dropwise addition of 0.3 N KHSO₄ solution (100 mL). The resultant mixture was transferred to a separatory funnel adding sufficient 5% KHSO₄ solution (75 mL) to dissolve the solids. The organic phase was separated and the combined aqueous washes back-extracted with ether (100 mL). The combined ether extracts were washed with saturated NaCl solution, dried over anhydrous sodium sulfate and concentrated in vacuo with minimal heating. TLC (ethyl acetate): streaky spot (UV and PMA) Rf=0.48. TLC (methanol/methylene chloride, 1:9) major spot (UV and PMA): Rf=0.75.

The crude aldehyde was immediately taken up in aqueous ethanol (45 mL water/105 mL alcohol), placed in an ice bath and treated with sodium acetate (3.82 g, 46.6 mmol) and semicarbazide hydrochloride (5.20 g, 46.6 mmol, Aldrich). The mixture was stirred at 0° C. (ice bath) under a nitrogen atmosphere for 3 hrs, allowed to warm to room temperature, and stirred overnight (16 hrs). Most of the ethanol was removed under vacuum and the residue partitioned between ethyl acetate and water (100 mL each). The organic phase was washed sequentially with 5% KHSO₄, saturated sodium bicarbonate and saturated sodium chloride solutions; dried over anhydrous sodium sulfate and evaporated to dryness. The crude product of this reaction was combined with that of two similar procedures starting with 15.40 g and 4.625 g of N-(benzyloxycarbonyl)-L-(N′-methyl-N′-methoxy)aspartamide-β-(tert-butyl ester) (total: 35.525 g, 97 mmol) and these combined products were purified by flash chromotagraphy on silica gel eluting with acetone/methylene chloride (3:7) then methanol-acetone-methylene chloride (0.5:3:7) to give pure title product (27.73 g, 78.5%) as a colorless foam. TLC (MeOH—CH₂Cl₂, 1:9): single spot (UV and PMA), Rf=0.51.

Part C: (3S)-Amino-4-Oxobutanoic Acid tert-Butyl Ester Semicarbazone p-Toluenesulfonate Salt

To a solution of (3S)-(benzyloxycarbonyl)amino-4-oxobutanoic acid tert-butyl ester semicarbazone (13.84 g, 38.0 mmol) in absolute ethanol (250 mL) was added 10% Pd/C (1.50 g, Aldrich) and the resulting mixture stirred under an atmosphere of hydrogen (balloon) until TLC (methanol/methylene chloride, 1:9) indicated complete consumption of the starting material (60 min). Note: It is important to follow this reaction closely since the product can be over-reduced. The mixture was filtered though Celite and evaporated to an oil. The oil was chased with methylene chloride (2×75mL) then with methylene chloride/toluene (1:1, 75 mL) to give the crude amine as a white crystalline solid. TLC (EtOAc-pyridine-AcOH—H₂O; 60:20:5:10) single spot (UV and PMA) Rf=0.24. Note: In this TLC system, any over-reduced product will show up immediately below the desired product, Rf=0.18 (PMA only).

The crude amine was taken up in CH₃CN (60 mL) and treated with a solution of p-toluenesulfonic acid monohydrate (7.22 g, 38.0 mmol) in acetonitrile (60 mL). The crystalline precipitate was collected, washed with acetonitrile and ether, and air-dried to give the title compound (13.95 g, 92% yield) as a white, crystalline solid.

The optical purity of this material was checked by conversion to the corresponding Mosher amide [1.05 equiv (R)-(−)-α-methoxy-α-(trifluoromethyl)phenylacetyl chloride, 2.1 equivalents of i-Pr₂NEt in CH₂Cl₂, room temperature, 30 min]. The desired product has a doublet at 7.13 ppm (1H, d, J=2.4 Hz, CH═N) while the corresponding signal for its diastereomer is at 7.07 ppm. The optical purity of the title compound obtained from the above procedure is typically>95:5.

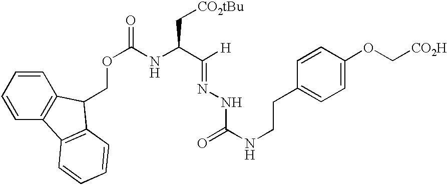

Preparation of (3S)-3-(9-Fluorenylmethoxycabonyl)Amino-4-Oxobutanoic Acid tert-Butyl Ester Semicarbazonyl-4-[2′-(4-Ethyl-Phenoxyacetic Acid)]

Part A: 4-[2′-(N-t-Butoxycarbonyl)Aminoethyl]Phenoxyacetic Acid, Methyl Ester

To a suspension 4-hydroxy-phenethylamine (7.00 g, 51.1 mmol, Aldrich) in dry dimethylformamide (50 mL) at room temperature under nitrogen was added di-tert-butyl dicarbonate (11.0 g, 50.5 mmol). After stirring at room temperature for 1 hrs, the resulting clear solution was treated with methyl bromoacetate (7.5 mL, 79 mmol) and cesium carbonate (17.5 g, 53.7 mmol). After stirring at room temperature for 16 hrs, TLC (Et₂O-toluene; 2:8) shows some unalkylated material remained (Rf=0.43) and a second portion of methyl bromoacetate (2.0 mL, 21 mmol) and cesium carbonate (4.5 g, 14 mmol) were added. After stirring for an additional 24 hrs, the mixture was partitioned between EtOAc-water (250 mL each), organic phase washed succesively with water (3×), 5% potassium bisulfate and saturated NaCl solutions, dried over anhydrous Na₂SO₄ and evaporated to dryness. Trituration of the residue with hexane gave 15.87 g of a tan solid. Filtration of the crude product through a pad of silica gel eluting with EtOAc-hexane (2:8) and crystallization from hexane gave the title compound (14.75, 93%) as a white granular, crystalline solid. TLC (Et₂O-toluene; 2:8) Rf=0.53.

Part B: 4-(2′-Aminoethyl)Phenoxyacetic Acid Methyl Ester, Hydrochloride

To a solution 4-[2′-(N-t-butoxycarbonyl)aminoethyl]phenoxyacetic acid, methyl ester (18.31 g, 59.3 mmol) in dioxane (55 mL) at room temperature was added 4.0 N HCl in dioxane (55 mL). After stirring at room temperature for 16 hrs, the mixture was diluted with Et₂O, the precipatate collected, washed throughly with Et₂O and dried in vacuo to give the title compound (14.55 g, 94%) was a fluffy white, crystalline solid.

Part C: 1-tert-Butoxycarbonyl-Semicarbazidyl-4-[2′-(4-Ethyl-Phenoxyacetic Acid)]Methyl Ester

A solution of t-butyl carbazate (6.60 g, 50 mmol) in dimethylformamide (50 mL) was added dropwise to a solution carbonyldiimidazole (8.10 g, 50 mmol) in dimethylformamide (80 mL) over 40 min at room temperature under nitrogen. After stirring at room temperature for an additional 30 min, 4-(2′-aminoethyl)phenoxyacetic acid, methyl ester, hydrochloride (12.3 g, 50 mmol) was added as a solid in one portion followed by a triethylamine (8.0 mL, 58 mmol) added dropwise over 30 min. After stirring at room temperature for 18 hrs, the mixture was partitioned between EtOAc-water (300 mL each). The organic phase was washed succesively with water (3×), 5% potassium bisulfate, saturated sodium bicarbonate, and saturated NaCl solutions, dried over anhydrous Na₂SO₄ and evaporated to dryness. Crystallization of the residue from EtOAc-hexane gave the title compound (15.50, 84%) as an off-white crystalline solid. TLC (MeOH—CH₂Cl₂; 1:9) Rf=0.45.

Part D: 1-tert-Butoxycarbonyl-Semicarbazidyl-4-[2′-(4-Ethyl-Phenoxyacetic Acid)]

A solution of 1-tert-butoxycarbonyl-semicarbazidyl-4-[2′-(4-ethyl-phenoxyacetic acid)]methyl ester (14.68 g, 40 mmol) in dioxane (50 mL) at room temperature under nitrogen was added 1.0 N LiOH solution (50 mL). After stirring at room temperature for 1 hrs, the mixture was acidified with conc. HCl and extracted with EtOAc (100 mL). The organic phase was washed with saturated NaCl solution, dried over anhydrous Na₂SO₄ and evaporated to a white solid. Recrystallization of the crude product from THF-EtOAc-hexane gave the title compound (13.44, 95%) as a white crystalline solid. TLC (AcOH—MeOH—CH₂Cl₂; 1:1:8)Rf=0.31.

Part E: Semicarbazidyl-4-[2′-(4-Ethyl-Phenoxyacetic Acid)]Hydrochloride

To a solution of 1-tert-butoxycarbonyl-semicarbazidyl-4-[2′-(4-ethyl-phenoxyacetic acid)] (13.43 g, 38.0 mmol) in dioxane (80 mL)-anisole (15 mL) at room temperature was added 4.0 N HCl in dioxane (35 mL). After stirring at room temperature for 18 hrs, additional 4.0 N HCl in dioxane (15 mL) was added. After an additional 6 hrs, the precipatate was collected, washed throughly with dioxane then Et₂O and dried in vacuo to give the title compound (11.67 g, 100%) was a white, crystalline solid.

Part F: N-(9-Fluorenylmethoxycarbonyl)-L-(N′-Methyl-N′-Methoxy)aspartamide β-(tert-Butyl)Ester

To a solution of N-(9-fluorenylmethoxycarbonyl)-L-aspartic acid-β-(tert-butyl)ester (16.48 g, 40 mmol) in CH₂Cl₂ (80 mL)-tetrahydrofuran (20 mL) at 0° C. (ice bath) under a nitrogen atmosphere was added 1-hydroxybenzotriazole hydrate (7.12 g, 46.5 mmol) followed by 1-ethyl-3-(3′,3′-dimethyl-1′-aminopropyl)carbodiimide hydrochloride (9.20 g, 48 mmol). After stirring at 0° C. for 15 min., N,O-dimethylhydroxylamine hydrochloride (4.68 g, 48 mmol) and N-methylmorpholine (5.2 mL, 47 mmol) were added. The mixture was allowed to warm to room temperature over 2 hours then stirred at room temperature for 16 hours. The solution was concentrated under vacuum and the residue partitioned between ethyl acetate-5% KHSO₄ (200 mL each). The organic phase was washed succesively with 5% KHSO₄, saturated sodium bicarbonate and saturated sodium chloride solutions; dried over anhydrous sodium sulfate and evaporated to an oil. Purification of the crude product by flash chromatography on silica gel eluting with EtOAc-hexane (30:70 then 35:65) gave the title product (17.75 g, 98% yield) as a colorless foam.

TLC (EtOAc-hexane; 1:1) Rf=0.35.

Part G: (3S)-3-(9-Fluorenylmethoxycabonyl)Amino-4-Oxobutanoic Acid tert-Butyl Ester Semicarbazonyl-4-[2′-(4-Ethyl-Phenoxyacetic Acid)]

To a solution of N-(9-fluorenylmethoxycarbonyl)-L-(N′-methyl-N′-methoxy)aspartamide-β-(tert-butyl)ester (13.20 g, 29 mmol) in anhydrous ether (250 mL) at 0° C. (ice bath) under a nitrogen atmosphere was added dropwise to a 1.0 M solution of LiAlH₄ in ether (14.5 mL, 14.5 mmol) at such a rate as to keep the reaction solution temperature between 0-5° C. (addition time 15-20 min). After the addition of the lithiumn aluminum hydride reagent was complete, the mixture was stirred at 0-5° C. for 1 hrs, then quenched by the dropwise addition of 0.3 N KHSO₄ solution (100 mL). After adding sufficient 0.3 N KHSO₄ solution to dissolve most of the inorganic salts, the mixture was transferred to a seperatory funnel. The organic phase was separated and the aqueous phase back-extracted with ether (100 mL). The combined ether extracts were washed with saturated NaCl solution, dried over anhydrous sodium sulfate and concentrated in vacuo with minimal heating. TLC (EtOAc-hexane): Rf=0.40.

The crude aldehyde was immediately taken up in ethanol (105 mL)-water(45 mL)-tetrahydrofuran(75 mL), placed in an ice bath and treated with sodium acetate (3.20 g, 39 mmol) and semicarbazidyl-4-[2′-(4-ethyl-phenoxyacetic acid)]hydrochloride (8.65 g, 30 mmol). The mixture was stirred at 0° C. (ice bath) under a nitrogen atmosphere for 3 hrs, allowed to warm to room temperature, and stirred overnight (16 hrs). The mixture was concentrated on a rotovap, diluted with water and resulting precipitate collected by suction. The material was dried in vacuo to give 18.36 g of crude product as a white solid. The crude product of this reaction was combined with that of a smaller scale reaction (6.34 g) starting with 4.55 g (10 mmol) of N-(9-fluorenylmethoxycarbonyl)-L-(N′-methyl-N′-methoxy)aspartamide-β-(tert-butyl ester) and partitioned between ethyl acetate-tetrahydrofuran(1:1) and 5% KHSO₄. The organic phase was washed with 5% KHSO₄ and saturated sodium chloride solutions, dried over anhydrous sodium sulfate and evaporated to dryness. The residue was purified by filtration through a pad of silica gel eluting with terahydrofuran/methylene chloride (1:1). The combined product-containing fractions were evaporated to dryness and recrystallized from tetrahydrofuran-Et₂O to give pure title product (17.01 g, 69%) as a white solid. TLC (AcOH—MeOH—CH₂Cl₂, 1:1:40): Rf=0.19.

Assay for Inhibition of ICE/ced-3 Protease Family Activity

A. Determination of IC₅₀ Values

Fluorescence enzyme assays detecting the activity of the compounds of Formula I utilizing the recombinant ICE and CPP32 enzymes are performed essentially according to Thomberry et al. (Nature, 356:768:774 (1992)) and Nicholson et al. (Nature, 376:37-43 (1995)) respectively, (herein incorporated by reference) in 96 well microtiter plates. The substrate is Acetyl-Tyr-Val-Ala-Asp-amino-4-methylcoumarin (AMC) for the ICE assay and Acetyl-Asp-Glu-Val-Asp-amino-4-methylcoumarin for the CPP32, Mch2, Mch3 and Mch5 assays. Enzyme reactions are run in ICE buffer (25 mM HEPES, 1 mM EDTA, 0.1% CHAPS, 10% sucrose, pH 7.5) containing 2 mM DTT at room temperature in duplicate. The assays are performed by mixing the following components:

50 μL ICE, Mch2, Mch5, CPP32 (18.8, 38, 8.1 and 0.153 nM concentrations, respectively) or Mch3 (1 unit) enzyme in ICE buffer containing either 8.0 (ICE, Mch2, Mch3, CPP32) or 20 (Mch5) mM DTT;

50 μL compound of Formula 1 or ICE buffer (control); and

100 μL of 20 μM substrate.

The enzyme and the compound of Formula I to be assayed are allowed to preincubate in the microtitre plate wells for 30 minutes at room temperature prior to the addition of substrate to initiate the reaction. Fluorescent AMC product formation is monitored for one hour at room temperature by measuring the fluorescence emission at 460 nm using an excitation wavelength of 360 nm. The fluorescence change in duplicate (control) wells are averaged and the mean values are plotted as a function of inhibitor concentration to determine the inhibitor concentration producing 50% inhibition (IC₅₀).

B. Determination of the Dissociation Constant Ki and Irreversible Rate Constant k₃ for Irreversible Inhibitors

For the irreversible inhibition of a ICE/ced-3 Family Protease enzyme with a competitive irreversible inhibitor; using the model represented by the following formulas:

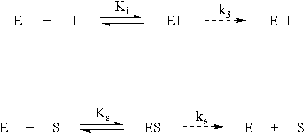

The product formation at time t may be expressed as:

$${[ P ]}_{t} = {[ E ]}^{T} ( \frac{[ S ] {K}_{i}}{[ I ] {K}_{s}} ) ( \frac{{k}_{s}}{{k}_{3}} ) [ 1 - {ⅇ}^{- {k}_{3} t / ( 1 + \frac{{K}_{i}}{[ I ]} ( 1 + \frac{[ S ]}{{K}_{s}} ) )} ] \qquad Equation 1$$

where E, I, EI and E-I denote the active enzyme, inhibitor, non-covalent enzyme-inhibitor complex and covalent enzyme-inhibitor adduct, respectively. The Kᵢ value is the overall dissociation constant of the reversible binding steps, and k₃ is the irreversible rate constant. The [S] and Kₛ values are the substate concentration and dissociation constant of the substrate bound to the enzyme, respectively. [E]^{T} is the total enzyme concentration.

The above equations may be used to determine the Kᵢ and k₃ values of a given inhibitor bound to a ICE/ced-3 family protease. Thus, a continuous assay may be run for sixty minutes at various concentrations of the inhibitor and the substrate. The assay may be formulated essentially the same as described above, except that the reaction is initiated by adding the enzyme to the substrate-inhibitor mixture. The Kᵢ and k₃ values are obtained by simulating the product AMC formation as a function of time according to Equation 1.

The following are examples of compounds of the invention.

### EXAMPLE 1

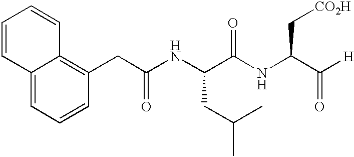

(3S)-3-[N-((1-Naphthyl)Acetyl)Leucinyl]Amino-4-Oxobutanoic Acid

Part A: (3S)-3-[(N-Benzyloxycarbonyl)Leucinyl]Amino-4-Oxobutanoic Acid tert-Butyl Ester Semicarbazone

To a solution of (N-benzyloxycarbonyl)leucine N-hydroxysuccinimde ester (5.0 mmol) in CH₂Cl₂ (30 mL) at room temperature under nitrogen is added (3S)-amino-4-oxobutanoic acid tert-butyl ester semicarbazone, p-toluenesulfonate salt (6.4 mmol) followed by diisopropyl ethylamine (1.2 mL, 6.9 mmol). After stirring at room temperature for 16 hrs, the mixture is concentrated and the residue partitioned between EtOAc-5% KHSO₄. The organic phase is washed with 5% KHSO₄, saturated NaHCO₃ and saturated NaCl solutions, dried over anhydrous Na₂SO₄ and evaporated to give the title compound.

Part B: (3S)-3-(Leucinyl)Amino-4-Oxobutanoic Acid tert-Butyl Ester Semicarbazone

To a solution of crude (3S)-[(N-benzyloxycarbonyl)leucinyl]amino-4-oxobutanoic acid tert-butyl ester semicarbazone (ca 5.0 mmol) in absolute EtOH (40 mL) is added 10% Pd—C (0.40 g) and resulting mixture is stirred under a hydrogen atmosphere (balloon) for 1.5 hrs. The mixture is filtered through Celite washing the filter cake with CH₂Cl₂ and the combined filtrates evaporated to dryness. The residue is chased with CH₂Cl₂ (2×20 mL) to give the title product.

Part C: (3S)-3-[N-((1-Naphthyl)Acetyl)Leucinyl]Amino-4-Oxobutanoic Acid tert-Butyl Ester Semicarbazone

To a solution of (1-naphthyl)acetic acid (0.74 mmol) and (3S)-3-(leucinyl)amino-4-oxobutanoic acid tert-butyl ester semicarbazone (0.83 mmol) in N-methylpyrrolidone(2.0 mL)-CH₂Cl₂(2.0 mL) at 0° C. (ice bath) under nitrogen is added hydroxybenzotriazole hydrate (0.130 g) followed by 1-ethyl-3-(3′,3′-dimethyl-1′-aminopropyl)carbodiimide hydrochloride (1.02 mmol). After stirring at 0° C. for 1 hr and at room temperature for 5 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The crude product is purified by flash chromatography eluting with MeOH—CH₂Cl₂ (2:100 then 5:100) to give the title compound.

Part D: (3S)-3-[N-((1-Naphthyl)Acetyl)Leucinyl]Amino-4-Oxobutanoic Acid Semicarbazone

To a solution of (3S)-3-[N-((1-naphthyl)acetyl)leucinyl]amino-4-oxobutanoic acid tert-butyl ester semicarbazone (0.69 mmol) in CH₂Cl₂(2.0 mL)-anisole(0.5 mL) at room temperature under nitrogen is added trifluoroacetic acid (2.0 mL). The resulting solution is stirred at room temperature for 3 hrs, evaporated to dryness and chased with toluene-CH₂Cl₂ (1:1). The residue is triturated with Et₂O to give the title compound (100%).

Part E: (3 S)-3-[N-((1-Naphthyl)Acetyl)Leucinyl]Amino-4-Oxobutanoic Acid

A solution of (3S)-3-[N-((1-naphthyl)acetyl)leucinyl]amino-4-oxobutanoic acid semicarbazone (0.68 mmol) in 37% aqueous formaldehyde(1.0 mL)-acetic acid(1.0 mL)-methanol(3.0 mL) is stirred at room temperature under nitrogen for 3.5 hrs. The resulting solution is diluted with water and extracted with EtOAc. The extract is washed with water and saturated NaCl solution, dried over anhydrous Na₂SO₄ and evaporated to dryness. The residue is taken up in EtOAc, filtered through Celite and evaporated to dryness. The product is taken up in a small amount of dioxane, diluted with water, frozen and lyophilized to give the title compound (79%).

### EXAMPLE 2

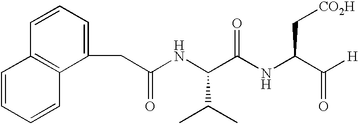

(3S)-3-[N-((1-Naphthyl)Acetyl)Valinyl]Amino-4-Oxobutanoic Acid

Part A: (3S)-3-[(N-Benzyloxycarbonyl)Valinyl]Amino-4-Oxobutanoic Acid tert-Butyl Ester Semicarbazone

To a solution of (N-benzyloxycarbonyl)valine (8.10 mmol) in CH₂Cl₂(80 mL) at 0° C. (ice bath) under nitrogen is added hydroxybenzotriazole hydrate followed by 1-ethyl-3-(3′,3′-dimethyl-1′-aminopropyl)carbodiimide hydrochloride (12.2 mmol). After stirring at 0° C. for 10 min, (3S)-amino-4-oxobutanoic acid tert-butyl ester semicarbazone, p-toluenesulfonate salt (8.10 mmol) followed by N-methylmorpholine (0.89 mL, 8.10 mmol) is added. After stirring at 0° C. for 2 hrs and at room temperature for 20 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The crude product is purified by flash chromatography eluting with MeOH—CH₂Cl₂ (2:100 then 5:100) to give the title compound (93%).

Part B: (3S)-3-[N-((1-Naphthyl)Acetyl)Valinyl]Amino-4-Oxobutanoic Acid

Starting with (3S)-3-[(N-benzyloxycarbonyl)valinyl]-amino-4-oxobutanoic acid tert-butyl ester semicarbazone and following the general method described in Example 1, Parts B through E, the title compound is also prepared.

### EXAMPLE 3

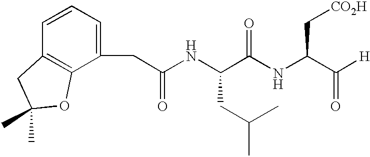

(3S)-3-[N-((2,3-Dihydro-2,2-Dimethyl-Benzofuranyl)Acetyl)Leucinyl]Amino-4-Oxobutanoic Acid

Part A: (3S)-3-[N-(9-Fluorenylmethoxycabonyl)Leucinyl]Amino-4-Oxobutanoic Acid tert-Butyl Ester Semicarbazonyl-4-[2′-(4-Ethyl-Phenoxyacetyl)]Aminomethylpolystrene

Aminomethylpolystryene resin (10.0 g, 100-200 mesh, 0.71 meq/g) is placed in a 200 mL filter tube equipped with a vacuum stopcock and glass frit and washed successively with CH₂Cl₂(50 mL)/dimethylformamide(50 mL), diisopropylethylamine(5 mL)/dimethylformamide(30 mL), dimethylformamide (2×40 mL) and tetrahydrofuran (30 mL). The resin is suspended in tetrahydrofuran(20 mL)/N-methylpyrolidinone(20 mL) with nitrogen agitation through the bottom of the frit and treated with diiospropylethylamine (1.9 mL, 10.9 mmol) and (3S)-3-(9-fluorenylmethoxycarbonyl)amino-4-oxobutanoic acid tert-butyl ester semicarbazonyl-4-[2′-(4-ethyl-phenoxyacetic acid)] (2.24 g, 3.56 mmol). After all of the solid has dissolved (approx. 10 min), the mixture is treated with pyBOP [benzotriazolyloxy-tris(N-pyrolidinyl)phosphonium hexafluorophosphate, 2.78 g, 5.34 mmol) in one portion. After mixing by nitrogen agitation for 3 hrs, the supernatant is removed by suction and the resin washed succesively with tetrahydrofuran (2×50 mL), dimethylformamide (3×50 mL) and CH₂Cl₂ (2×50 mL). Unreacted amine groups are capped by treatment with a mixture of acetic anhydride(10 mL)/dimethylformamide(30 mL)/diisopropylethylamine(1.0 mL). After mixing by nitrogen agitation for 1 hrs, the supernatant is removed by suction and the resin washed with dimethylformamide(4×50 mL).

The resin is treated with piperidine(10 mL)/dimethylformamide(40 mL) and mixed by nitrogen agitation for 45 min. The supernatant is removed by suction and the resin washed with dimethylformamide(4×50 mL) and tetrahydrofuran (50 mL).

The resin is suspended in tetrahydrofuran(20 mL)/N-methylpyrolidinone(20 mL), treated with N-(9-fluorenylmethoxycabonyl)leucine (2.52 g, 7.12 mmol), diisopropylethylamine (3.8 mL, 21.8 mmol) and pyBOP (5.56 g, 10.7 mmol) and mixed by nitrogen agitation for 2.5 hrs. The supernatant is removed by suction and the resin washed successively with dimethylformamide (3×40 mL) and CH₂Cl₂ (3×40 mL), methanol (2×40 mL) and Et₂O (2×40 mL). The resin is dried in vacuo to give the title product. Based on the starting semicarbazone-acid, the resin loading may be calculated as approximately 0.27 meq/g.

Part B: (3S)-3-[N-((2,3-Dihydro-2,2-Dimethyl-7-Benzofuranyl)Acetyl)Leucinyl]Amino-4-Oxobutanoic Acid

An aliquot of the Part A resin (ca 0.032 mmol) is placed in a 6 mL Supelco™ fitration tube equipped with a 20 μm polyethylene frit, treated with piperidine-dimethylformamide (1.0 mL, 1:4 v/v) and mixed on an orbital shaker for 1 hrs. The supernatant is removed by suction and the resin washed with dimethylformamide (4×1.0 mL) and CH₂Cl₂ (3×1.0 mL). The resin is treated with 0.5M iPr₂NEt in N-methylpyrolidinone (0.40 mL, 0.20 mmol), (2,3-dihydro-2,2-dimethyl-7-benzofuranyl)acetic acid (0.12 mmol) and 0.25M pyBOP in N-methylpyrolidinone (0.40 mL, 0.10 mmol). The mixture is mixed on an orbital shaker under an nitrogen atmosphere for 16 hrs. The supernatant is removed by suction and the resin washed succesively with dimethylformamide (3×1.0 mL) and CH₂Cl₂ (3×1.0 mL), methanol (2×1.0 mL) and Et₂O (2×1.0 mL).

The resin is treated with 1.0 mL of CH₂Cl₂ and allowed to re-swell for 15 min. The solvent is removed by suction and the resin treated with trifluoroacetic acid-CH₂Cl₂-anisole (1.0 mL, 4:3:1 v/v/v). After mixing on an orbital shaker under nitrogen for 6 hrs, the supernatant is removed by suction and the resin washed with CH₂Cl₂ (4×1.0 mL). The resin is treated with 37% aqueous formaldehyde-acetic acid-tetrahydrofuran-trifluoroacetic acid (1.0 mL, 1:1:5:0.025 v/v/v/v) and mixed on an orbital shaker under nitrogen for 4 hrs. The supernatant is collected by suction, the resin washed with tetrahydrofuran (3×0.5 mL). The combined filtratesare blown down under nitrogen. The residue is taken up in methanol (0.5 mL), filtered and applied directly to a 3 mL Supelco™ LC-18 reverse phase extraction tube which has been pre-conditioned with water, and eluted successively with 3 mL each of 10% MeOH-water, 30% MeOH-water, 60% MeOH-water and 90% MeOH-water. The product-containing fractions (TLC) are combined and evaporated to dryness to give the title compound.

### EXAMPLES 4-36

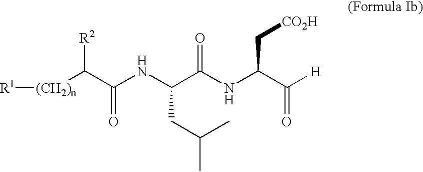

Following the general procedure set forth in Example 3, Part B; the compounds of Formula Ib (Examples 4 through 36) shown in Table 1 below may also be prepared.

TABLE 1

| Ex. | | | |
|---|---|---|---|
| No. | R¹ | n | R² |
| 4 | 1-naplthyl | 0 | H |
| 5 | 2-naphthyl | 0 | H |
| 6 | 1-naphthyl | 0 | CH₃ |
| 7 | 6-Br-1-naphthyl | 0 | CH₃ |
| 8 | 2-naphthyl | 1 | H |
| 9 | 1-naphthyl | 1 | H |
| 10 | 2-Me-1-naphthyl | 0 | H |
| 11 | 4-MeO-1-naphthyl | 0 | H |
| 12 | 4-Cl-1-naphthyl | 0 | H |
| 13 | 2,4-diCl-1-naphthyl | 0 | H |
| 14 | 1-isoquinolinyl | 0 | H |
| 15 | 4-quinolinyl | 0 | H |
| 16 | 5-quinolinyl | 0 | H |
| 17 | 5-isoquinolinyl | 0 | H |
| 18 | 8-quinolinyl | 10 | H |
| 19 | phenyl | 0 | H |
| 20 | phenyl | 0 | CH₃ |
| 21 | phenyl | 1 | H |
| 22 | phenyl | 1 | CH₃ |
| 23 | 2-biphenyl | 0 | H |
| 24 | 3-biphenyl | 0 | H |
| 25 | 4-biphenyl | 0 | H |
| 26 | (2-benzyl)phenyl | 0 | H |
| 27 | (4-benzyl)phenyl | 0 | H |
| 28 | (4-phenoxy)phenyl | 0 | H |
| 29 | (2-benzyloxy)phenyl | 0 | H |
| 30 | (4-benzyloxy)phenyl | 0 | H |
| 31 | (2-cyclo-pentyl)- | 0 | H |
| | phenyl | | |
| 32 | (4-cyclo-pentyl)- | 0 | H |
| | phenyl | | |
| 33 | [2-(1-adamantanyl)- | 0 | H |
| | 4-Me]phenyl | | |
| 34 | 4-(1-adamantanyl)- | 0 | H |
| | phenyl | | |
| 35 | 5,6,7,8-tetrahydro-1- | 0 | H |
| | naphthyl | | |
| 36 | 5,6,7,8-tetrahydro-2- | 0 | H |
| | naphthyl | | |

### EXAMPLE 37

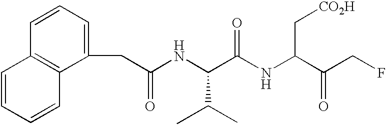

(3RS)-3-[N-((1-Naphthyl)Acetyl)Valinyl]Amino-5-Fluoro-4-Oxopentanoic Acid

Part A: (3RS,4RS)-3-[(N-Benzyloxycarbonyl)Valinyl]Amino-5-Fluoro-4-Hydroxypentanoic Acid, tert-Butyl Ester

To a solution of (N-benzyloxycarbonyl)valine (0.322 g, 1.32 mmol) in CH₂Cl₂(7.0 mL) at 0° C. (ice bath) under nitrogen is added hydroxybenzotriazole hydrate (0.219 g) followed by 1-ethyl-3-(3′,3′-dimethyl-1′-aminopropyl)carbodiimide hydrochloride (0.319 g, 1.65 mmol). After stirring at 0° C. for 10 min, the mixture is treated with (3RS,4RS)-3-amino-5-fluoro-4-hydroxypentanoic acid, tert-butyl ester (0.228 g, 1.1 mmol, prepared as described in Tetrahedron Letters 1994,35, 9693-9696) and the reacton allowed to warm to room temperature. After stirring at room temperature for 24 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The residue is purified by flash chromatography eluting with EtOAc-hexane (1:1) to give the title compound.

Part B: (3RS,4RS)-3-(Valinyl)Amino-5-Fluoro-4-Hydroxypentanoic Acid, tert-Butyl Ester

To a solution of (3RS,4RS)-3-[(N-benzyloxycarbonyl)valinyl]amino-5-fluoro-4-hydroxypentanoic acid, tert-butyl ester (1.00 g, 2.30 mmol) in EtOH (130 mL) is added 10% Pd—C (0.120 g) and resulting mixture stirred under a hydrogen atmosphere (balloon) for 1 hrs. The mixture is filtered through Celite washing the filter cake with CH₂Cl₂ and the combined filtrates evaporated to dryness. The residue is chased with CH₂Cl₂ to give the title product.

Part C: (3RS,4RS)-3-[N-((1-Naphthyl)Acetyl)Valinyl]Amino-5-Fluoro-4-Hydroxypentanoic Acid, tert-Butyl Ester

To a solution of (1-naphthyl)acetic acid (1.0 mmol) in dimethylformamide(4.0 mL)-CH₂Cl₂(6.0 mL) at 0° C. (ice bath) under nitrogen is added hydroxybenzotriazole hydrate (0.168 g) followed by 1-ethyl-3-(3′,3′-dimethyl-1′-aminopropyl)carbodiimide hydrochloride (0.249 g, 1.3 mmol). After stirring for 10 min, the mixture is treated with a solution of (3RS,4RS)-3-(valinyl)amino-5-fluoro-4-hydroxypentanoic acid, tert-butyl ester (0.319 g, 1.04 mmol) in CH₂Cl₂(8.0 mL). After stirring at 0° C. for 1 hrs and at room temperature for 3 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The residue is purified by flash chromatography on silica gel eluting with EtOAc-hexane (3:2) to give the title compound.

Part D: (3RS)-3-[N-((1-Naphthyl)Acetyl)Valinyl]Amino-5-Fluoro-4-Oxopentanoic Acid, tert-Butyl Ester

To a solution of (3RS,4RS)-3-[N-((1-naphthyl)acetyl)valinyl]amino-5-fluoro-4-hydroxypentanoic acid, tert-butyl ester (0.315 mmol) and N-methylmorpholine N-oxide (0.144 g, 0.98 mmol) in CH₂Cl₂ (5.0 mL) at room temperature is added activated 4 Å molecular sieves. After stirring at room temperature for 20 min, the mixture is treated with tetra(n-propyl)ammonium perruthenate (0.011 g). After stirring at room temperature for 3.5 hrs, the mixture may be filtered through Celite and evaporated to dryness. The residue is purified by flash chromatography on silica gel eluting with EtOAc-hexane (3:4) to give the title compound.

Part E: (3RS)-3-[N-((1-Naphthyl)Acetyl)Valinyl]Amino-5-Fluoro-4-Oxopentanoic Acid

To a solution of (3RS)-3-[N-((1-naphthyl)acetyl)valinyl]amino-5-fluoro-4-oxopentanoic acid, tert-butyl ester (0.23 mmol) in CH₂Cl₂(2.0 mL)-anisole(0.5 mL) at room temperature under nitrogen is added trifluoroacetic acid (1.0 mL). The resulting clear solution is stirred at room temperature for 1 hrs, evaporated to dryness and chased with toluene-CH₂Cl₂ (1:1). The residue is purified by flash chromatography on silica gel eluting with AcOH—MeOH—CH₂Cl₂ (0.5:2:100) to give the title compound.

### EXAMPLES 38-40

Starting with (3RS,4RS)-3-(valinyl)amino-5-fluoro-4-hydroxypentanoic acid, tert-butyl ester (see Example 37, Part B) and following the methods described in Example 37, Parts C through E, the compounds shown below in Table 2 are also prepared:

TABLE 2

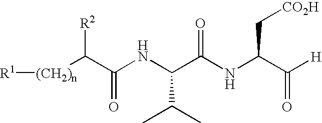

| Ex. | R¹ | n | R² |
|---|---|---|---|
| 38 | 2-naphthyl | 0 | H |
| 39 | 1-naphthyl | 1 | H |
| 40 | (2-PH)Ph | 0 | H |

### EXAMPLE 41

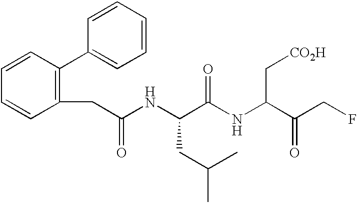

(3RS)-3-[N-((2-Phenylphenyl)Acetyl)Leucinyl]Amino-5-Fluoro-4-Oxopentanoic Acid

Part A: (3RS,4RS)-3-[(N-Benzyloxycarbonyl)Leucinyl]Amino-5-Fluoro-4-Hydroxypentanoic Acid, tert-Butyl Ester

To a solution of (3RS,4RS)-3-amino-5-fluoro-4-hydroxypentanoic acid, tert-butyl ester (0.230 g, 1.1 mmol) in CH₂Cl₂ (2.0 mL) at room temperature under nitrogen is added (N-benzyloxycarbonyl)leucine, N-hydroxysuccinimde ester (0.402 g, 1.1 mmol). After stirring at room temperature for 16 hrs, the mixture is evaporated to dryness and the residue purified by flash chromatography on silica gel eluting with EtOAc-hexane (1:2) to give the title compound.

Part B: (3RS,4RS)-3-(Leucinyl)Amino-5-Fluoro-4-Hydroxypentanoic Acid, tert-Butyl Ester, p-Toluenesulfonate Salt

To a solution of (3RS,4RS)-3-[(N-benzyloxycarbonyl)leucinyl]amino-5-fluoro-4-hydroxypentanoic acid, tert-butyl ester (0.332 g, 0.734 mmol) in MeOH (100 mL) is added p-toluenesulsufonic acid hydrate (0.140 g, 0.737 mmol) and 10% Pd—C (0.033 g) and resulting mixture stirred under a hydrogen atmosphere (balloon) for 2 hrs. The mixture is filtered through Celite washing the filter cake with CH₂Cl₂ and the combined filtrates evaporated to dryness. The residue is chased with CH₂Cl₂ to give the title product.

Part C: (3RS)-3-[N-((2-Phenylphenyl)Acetyl)Leucinyl]Amino-5-Fluoro-4-Oxopentanoic Acid

Starting with (3RS,4RS)-3-(leucinyl)amino-5-fluoro-4-hydroxypentanoic acid, tert-butyl ester, p-toluenesulfonate salt and following the methods described in Example 37, Parts C through E utilizing (2-phenylphenyl)acetic acid in place of (1-naphthyl)acetic acid in Part C, gives the title compound.

### EXAMPLE 42

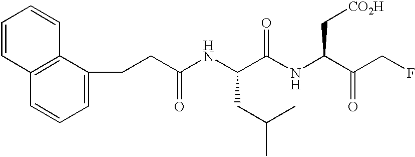

(3RS)-3-[N-(3-(1′-Naphthyl)Propionyl)Leucinyl]Amino-5-Fluoro-4-Oxopentanoic Acid

Starting with (3RS,4RS)-3-(leucinyl)amino-5-fluoro-4-hydroxypentanoic acid, tert-butyl ester, p-toluenesulfonate salt and following the methods described in Example 37, Parts C through E utilizing 3-(1′-naphthyl)propionic acid in place of (1-naphthyl)acetic acid in Part C, gives the title compound.

### EXAMPLE 43

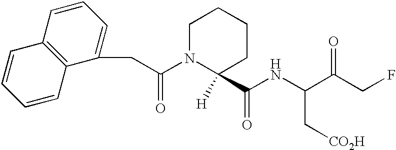

(S,3RS)-3-[N-(1-Naphthyl)Acetyl)Homoprolinyl]Amino-5-Fluoro-4-Oxopentanoic Acid

Following the general methods described in Example 37, Parts A through E, and utilizing N-(benzyloxycarbonyl)-homoproline in place of N-(benzyloxycarbonyl)valine in Part A, the title compound is also prepared.

### EXAMPLE 44

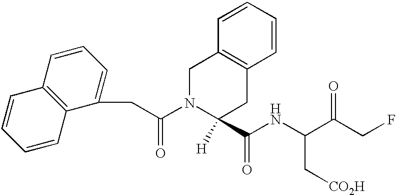

(2′S,3RS)-3-[N-(1-Naphthyl)Acetyl)-1,2,3,4-Tetrahydroisoquinoline-2′-Carbonyl]Amino-5-Fluoro-4-Oxopentanoic Acid

Following the general methods described in Example 37, Parts A through E, and utilizing (2S)—N-(benzyloxycarbonyl)-1,2,3,4-tetrahydroisoquinoline-2-carboxylic acid in place of N-(benzyloxycarbonyl)valine in Part A, the title compound is also prepared.

### EXAMPLE 45

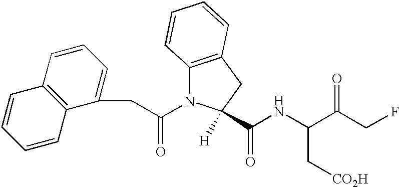

(2′S ,3RS)-3-[N-((1-Naphthyl)Acetyl)Indoline-2′-Carbonyl]Amino-5-Fluoro-4-Oxopentanoic Acid

Part A: (2S)—N-[(1-Naphthyl)Acetyl]Indoline-2′-Carboxylic Acid Methyl Ester

To a solution of (1-naphthyl)acetic acid (5.53 mmol) in ether (30 mL) at 0° C. is treated with phosphorus pentachloride (1.267 g, 6.08 mmol). After stirring at 0° C. for 20 min and at room temperature for 30 min, the mixture is evaporated to dyness and the residue chased with toluene (2×). The resulting crude acid chloride is taken up in toluene (10 mL) and added to a vigorously stirring mixture of methyl (S)-indoline-2-carboxylate hydrochloride (1.182 g, 5.53 mmol) in toluene (10 mL)/aqueous NaHCO₃ solution (2.1 g in 18 mL of H₂O) under N₂ at 0° C. The mixture is stirred for 30 min then partitioned between EtOAc and 5% KHSO₄. The organic phase is washed with 5% KHSO₄, sat'd NaHCO₃ (2×) and saturated NaCl solutions, dried (Na₂SO₄), and evaporated to dryness to give the title compound.

Part B: (2S)—N-[(1-Naphthyl)Acetyl]Indoline-2-Carboxylic Acid

To a solution of (2S)—N-[(1-naphthyl)acetyl]indoline-2-carboxylic acid methyl ester (2.77 mmol) in tetrahydrofuran (3.3 mL) at 0° C. is added 1.0 N LiOH solution (3.3 mL, 3.3 mmol). After stirring at 0° C. for 2 hours the mixture is concentrated, diluted with water, acidified to pH 3, and extracted with EtOAc. The EtOAc extract is washed with saturated NaCl, dried (Na₂SO₄), and evaporated to give the title compound.

Part C: (2′S,3RS,4RS)—N-[((1 -Naphthyl)Acetyl)Indoline-2′-Carbonyl]Amino-5-Fluoro-4-Hydroxypentanoic Acid t-Butyl Ester

To a solution of (2S)—N-[(1-naphthyl)acetyl]-indoline-2-carboxylic acid (0.8 mmol) in CH₂Cl₂ (2.0 mL)-dimethylformamide (0.5 mL) at 0° C. under nitrogen is added hydroxybenzotriazole hydrate (0.129 g) followed by 1-ethyl-3-(3′,3′-dimethyl-1′-aminopropyl)carbodiimide hydrochloride (0.184 g, 0.96 mmol). After stirring at 0° C. for 10 min, a solution of (3RS,4RS)-3-amino-5-fluoro-4-hydroxypentanoic acid, tert-butyl ester (0.166 g, 0.8 mmol) in CH₂Cl₂ (3.0 mL) is added. After stirring at 0° C. for 1 hrs and at room temperature for 3 hrs, the reaction mixture is partitioned between EtOAc and 5% KHSO₄. The organic phase is washed with 5% KHSO₄, saturated NaHCO₃ (2×) and saturated NaCl solutions, dried (Na₂SO₄), and evaporated to dryness to give the crude title compound.

Part D: (2′S,3RS)—N-[((1 -Naphthyl)Acetyl)Indoline-2′-Carbonyl]Amino-5-Fluoro-4-Oxopentanoic Acid t-Butyl Ester

To a solution of 2.0 M oxalyl chloride-CH₂Cl₂ (0.3 mL, 0.6 mmol) at −78° C. under nitrogen is added dimethylsulfoxide (0.09 mL, 1.2 mmol). After stirring at −78° C. for 10 min, a solution of (2′S,3RS,4RS)—N-[((1-naphthyl)acetyl)indoline-2′-carbonyl]amino-5-fluoro-4-hydroxypentanoic acid t-butyl ester (0.48 mmol) in dry CH₂Cl₂ (3.0 mL) is added dropwise. After stirring at −78° C. for 15 min, triethylamine (0.27 mL, 2.5 mmol) is added dropwise, the mixture stirred for 10 min, then allowed to warm to room temperature. After an additional 1 hr, the mixture is partitioned between EtOAc and 5% KHSO₄. The organic phase is washed with 5% KHSO₄ and saturated NaCl solutions, dried (Na₂SO₄), and evaporated to dryness. The crude product is purified by flash chromatography on silica gel eluting with EtOAc/hexane (1:2) to give the title compound.

Part E: (2′S,3RS)—N-[((1-Naphthyl)Acetyl)Indoline-2′-Carbonyl]Amino-5-Fluoro-4-Oxopentanoic Acid

To a solution of (2′S,3RS)—N-[((1-naphthyl)acetyl)indoline-2′-carbonyl]amino-5-fluoro-4-oxopentanoic acid t-butyl ester (0.20 mmol) in anisole (0.2 mL)-CH₂Cl₂ (2.0 mL) at room temperature under nitrogen is added trifluoroacetic acid (1.0 mL). After stirring at room temperature for 1.5 hrs, the mixture is concentrated then chased with CH₂Cl₂ and toluene. The residue is trituated with ether-hexane to give the title ccompound.

### EXAMPLE 46

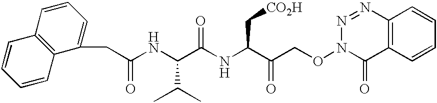

(3S)-3-[N-((1-Naphthyl)Acetyl)Valinyl]Amino-5-(1′,2′,3′-Benzotriazin-4′(3H)-on-3′-yloxy)-4-Oxopentanoic Acid

Part A: [(N-Benzyloxycarbonyl)Valinyl]Aspartic Acid β-tert-Butyl, α-Methyl Ester

To a solution of (N-benzyloxycarbonyl)valine (2.10 g, 8.36 mmol) in CH₂Cl₂(20 mL) at 0° C. (ice bath) under nitrogen is added hydroxybenzotriazole hydrate (1.74 g) followed by 1-ethyl-3-(3′,3′-dimethyl-1′-aminopropyl)carbodiimide hydrochloride (2.40 g, 12.5 mmol). After stirring at 0° C. for 10 min, the mixture is treated with aspartic acid, β-tert-butyl, α-methyl ester hydrochloride (2.00 g, 8.34 mmol) and N-methylmorpholine 1.1 mL, 10 mmol), and the reaction allowed to warm to room temperature. After stirring at room temperature for 2.5 hrs, the mixture is concentrated and the residue partitioned between EtOAc-water. The organic phase is washed with water, 5% KHSO₄, saturated NaHCO₃ and saturated NaCl solutions, dried over anhydrous Na₂SO₄ and evaporated to give the title compound.

Part B: N-(Valinyl)Aspartic Acid β-tert-Butyl α-Methyl Ester

To a solution of [(N-benzyloxycarbonyl)valinyl]aspartic acid, β-tert-butyl, α-methyl ester (2.14 g, 4.90 mmol) in EtOH (200 mL) is added 10% Pd—C (0.21 g) and resulting mixture stirred under a hydrogen atmosphere (balloon) for 2 hrs. The mixture is filtered through Celite washing the filter cake with CH₂Cl₂ and the combined filtrates evaporated to dryness. The residue is chased with CH₂Cl₂ to give the title product. The crude product may be used immediately for the next step.

Part C: [N-((1-Naphthyl)Acetyl)Valinyl]Aspartic Acid β-tert-Butyl, α-Methyl Ester

To a solution of (1-naphthyl)acetic acid (4.90 mmol) in CH₂Cl₂(45 mL) at 0° C. (ice bath) under nitrogen is added hydroxybenzotriazole hydrate (0.851 g) followed by 1-ethyl-3-(3′,3′-dimethyl-1′-aminopropyl)carbodiimide hydrochloride (1.33 g, 6.94 mmol). After stirring for 15 min, the mixture is treated with N-(valinyl)aspartic acid, β-tert-butyl, α-methyl ester (1.48 g, ca 4.90 mmol) and N-methylmorpholine (0.61 mL, 5.55 mmol). After stirring at 0° C. for 2 hrs and at room temperature for 16 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The residue is purified by flash chromatography on silica gel eluting with EtOAc-hexane (1:2) to give the title compound.

Part D: [N-((1-Naphthyl)Acetyl)Valinyl]Aspartic Acid, β-tert-Butyl Ester To a solution of [N-((1-naphthyl)acetyl)valinyl]aspartic acid, β-tert-butyl, α-methyl ester (3.87 mmol) in dioxane (9.0 mL)-water (3.0 mL) is added 1.0 N LiOH solution (4.3 mL, 4.3 mmol). After stirring at room temperature for 1 hr, the mixture is acidified with 1.0 N HCl and extracted with EtOAc. The extract is washed with saturated NaCl solution, dried over anhydrous Na₂SO₄ and evaporated to give the title compound.

Part E: (3S)-3-[N-((1-Naphthyl)Acetyl)Valinyl]Amino-5-Bromo-4-Oxopentanoic Acid tert-Butyl Ester

To a solution of [N-((1-naphthyl)acetyl)valinyl]aspartic acid, β-tert-butyl ester (8.40 mmol) and N-methylmorpholine (1.48 mL, 13.5 mmol) in tetrahydrofuran (37 mL) at −10° C. (NaCl/ice bath) under nitrogen is added isobutyl chloroformate (1.63 mL, 12.6 mmol). After stirring at −10° C. for 0.5 hrs, the mixture is filtered into another ice-cooled flask and the filter cake washed with cold tetrahydrofuran (approx. 15 mL). The resulting mixed anhydride solution is treated at −10° C. with excess diazomethane/Et₂O solution (prepared from 3.09 g, 21 mmol of 1-methyl-3-nitro-1-nitrosoguanidine, 15 mL 40% KOH/28 mL Et₂O). After stirring at −10° C. for 30 min and at room temperature for 15 min, the mixture is cooled to 0° C. (ice bath) and treated with 48% aqueous HBr (19.0 mL, 170 mmol). Gas evolution is observed. After 15 min, the mixture is partitioned between EtOAc-saturated NaHCO₃, the organic phase washed with saturated NaCl solution, dried over anhydrous Na₂SO₄ and evaporated. Trituration of the residue with Et₂O gives the title compound.

Part F: (3S)-3-[N-((1-Naphthyl)Acetyl)Valinyl]Amino-5-(1′,2′,3′-Benzotriazin-4′(3H)-on-3′-yloxy)-4-Oxopentanoic Acid, tert-Butyl Ester

To a solution of (3S)-3-[N-((1-naphthyl)acetyl)valinyl]amino-5-bromo-4-oxopentanoic acid tert-butyl ester (0.30 mmol) and 3-hydroxy-1,2,3-benzotriazin-4(3H)-one (0.059 g, 0.36 mmol) in dimethylformamide (2.0 mL) at room temperature under nitrogen is added potassium fluoride (0.061 g, 1.05 mmol). After stirring at room temperature for 5 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. Trituration of the residue with Et₂O-hexane gives the title compound.

Part G: (3S)-3-[N-((1-Naphthyl)Acetyl)Valinyl]Amino-5-(1′, 2′,3′-Benzotriazin-4′(3H)-on-3′-yloxy)-4-Oxopentanoic Acid

To a solution of (3S)-3-[N-((1-naphthyl)acetyl)valinyl]amino-5-(1′,2′,3′-benzotriazin-4′(3H)-on-3′-yloxy)-4-oxopentanoic acid, tert-butyl ester (0.23 mmol) in CH₂Cl₂(2.0 mL)-anisole(0.2 mL) at room temperature under nitrogen is added trifluoroacetic acid (1.0 mL). The resulting clear solution is stirred at room temperature for 2 hrs, evaporated to dryness and chased with toluene-CH₂Cl₂ (1:1). The residue is triturated with Et₂O-hexane to give the title compound.

### EXAMPLES 47-133

Starting with (3S)-3-[N-((1-naphthyl)acetyl)valinyl]amino-5-bromo-4-oxopentanoic acid tert-butyl ester (see Example 46, Part E) and following the methods described in Example 46, Parts F through G, the compounds shown below in Table 3 are also prepared:

TABLE 3

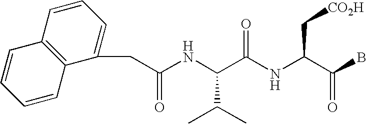

| Ex. | B |
|---|---|
| 47 | CH₂OCO(2,6-diCl-Ph) |
| 48 | CH₂Oph |
| 49 | CH₂O(2-F-Ph) |
| 50 | CH₂O(3-F-Ph) |
| 51 | CH₂O(4-F-Ph) |
| 52 | CH₂O(2,3-diF-Ph) |
| 53 | CH₂O(2,4-diF-Ph) |
| 54 | CH₂O(2,5-diF-Ph) |
| 55 | CH₂O(2,6-diF-Ph) |
| 56 | CH₂O(3,4-diF-Ph) |
| 57 | CH₂O(3,5-diF-Ph) |
| 58 | CH₂O(2,3,4-triF-Ph) |
| 59 | CH₂O(2,3,5-triF-Ph) |
| 60 | CH₂O(2,3,6-triF-Ph) |
| 61 | CH₂O(2,4,5-triF-Ph) |
| 62 | CH₂O(2,4,6-triF-Ph) |
| 63 | CH₂O(2,3,5,6-tetraF-Ph) |
| 64 | CH₂O(2,3,4,5,6-pentaF-Ph) |
| 65 | CH₂O(2-CF₃-Ph) |
| 66 | CH₂O(3-CF₃-Ph) |
| 67 | CH₂O(4-CF₃-Ph) |
| 68 | CH₂O(3,5-diCF₃-Ph) |
| 69 | CH₂O(2-F,3-CF₃-Ph) |
| 70 | CH₂O(2,6-diCl-Ph) |
| 71 | CH₂O(2-NO₂-Ph) |
| 72 | CH₂O(4-NO₂-Ph) |
| 73 | CH₂O(2-F,4-NO₂-Ph) |
| 74 | CH₂O(4-CN-Ph) |
| 75 | CH₂O(4-CF₃O-Ph) |
| 76 | CH₂O(4-H₂NCO-Ph) |
| 77 | CH₂O(4-PhCO-Ph) |
| 78 | CH₂O(4-Ph-Ph) |
| 79 | CH₂O(4-C₆F₅-2,3,5,6-tetraF-Ph) |
| 80 | CH₂O(4-PhO-Ph) |
| 81 | CH₂O[4-(4′-CF₃-PhO)Ph] |
| 82 | CH₂O(3-AcNH-Ph) |
| 83 | CH₂O(3,4-OCOS-Ph) |
| 84 | CH₂O(2-pyridinyl) |
| 85 | CH₂O(4,5-diCl-3-pyridazinyl) |
| 86 | CH₂O(2-naphthyl) |
| 87 | CH₂OPOPh₂ |
| 88 | CH₂OPO(Me)Ph |
| 89 | CH₂OPOMe₂ |
| 90 | CH₂OPO(n-hexyl)Ph |
| 91 | CH₂OPO(PhCH₂)Ph |
| 92 | CH₂OPO(Me)(4-F-Ph) |
| 93 | CH₂OPO(n-hexyl)(4-F-Ph) |
| 94 | CH₂OPO(Me)(1-naphthyl) |
| 95 | CH₂O(6-Me-2-pyron-4-yl) |
| 96 | CH₂O(4-coumarinyl) |
| 97 | CH₂O(2-Me-4-pyron-3-yl) |
| 98 | CH₂O[1,2-diMe-4(1H)-pyridon-3-yl] |
| 99 | CH₂O(3-flavonyl) |
| 100 | CH₂O(4,6-diMe-2-pyrimidinyl) |
| 101 | CH₂O(4-CF₃-2-pyrimidinyl) |
| 102 | CH₂S(4,6-diMe-2-pyrimidinyl) |
| 103 | CH₂O(2,6-diMe-4-pyrimidinyl) |
| 104 | CH₂O(6-CF₃-4-pyrimidinyl) |
| 105 | CH₂O(2-CF₃-4-pyrimidinyl) |
| 106 | CH₂S(2-imidazolyl) |
| 107 | CH₂S(1-Me-2-imidazolyl) |
| 108 | CH₂S(1H-1,2,4-triazol-3-yl) |
| 109 | CH₂S(4-Me-4H-1,2,4-triazol-3-yl) |
| 110 | CH₂S(1-Me-5-tetrazolyl) |
| 111 | CH₂S(1-Ph-5-tetrazolyl) |
| 112 | CH₂S(5-Me-1,3,4-thiadiazol-2-yl) |
| 113 | CH₂S(5-Ph-1,3,4-oxadiazol-2-yl) |
| 114 | CH₂S(3-Ph-1,2,4-oxadiazol-5-yl) |
| 115 | CH₂S(4-Ph-2-thiazolyl) |
| 116 | CH₂S(4,5-diPh-2-imidazolyl) |
| 117 | CH₂O(2-benzothiazolyl) |
| 118 | CH₂O(2-benzimidazolyl) |
| 119 | CH₂S(2-benzothiazolyl) |
| 120 | CH₂S(2-benzimidazolyl) |
| 121 | CH₂O(2-quinolinyl) |
| 122 | CH₂O(3-isoquinolinyl) |
| 123 | CH₂O(1-isoquinolinyl) |
| 124 | CH₂O(4-quinazolinyl) |
| 125 | CH₂O(8-quinolinyl) |
| 126 | CH₂O(3-Me-4-CO₂Et-isoxazol-5-yl) |
| 127 | CH₂O(1-Ph-3-CF₃-pyrazol-5-yl) |
| 128 | CH₂O(5-CO₂Me-isoxazol-3-yl) |
| 129 | CH₂O(5-iPr-isoxazol-3-yl) |
| 130 | CH₂O(3-benzoisoxazolyl) |
| 131 | CH₂O(1-Me-5-CF₃-pyrazol-3-yl) |
| 132 | CH₂O(1-benzotriazolyl) |
| 133 | CH₂O(N-phthalimidyl) |

### EXAMPLES 134-138

Starting from N-(valinyl)aspartic acid, β-tert-butyl, α-methyl ester (see Example 46, Part B), following the general methods described in Example 46, Parts C through G and utilizing (2-phenylphenyl)acetic acid in place of (1-naphthyl)acetic acid in Part C, and the appropriate acid or phenol in place of 3-hydroxy-1,2,3-benzotriazin-4(3H)-one in Part F, the compounds shown below in Table 4 may also be prepared:

TABLE 4

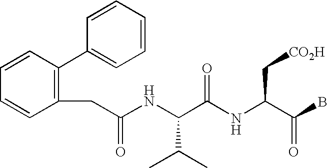

| Ex. | B |
|---|---|
| 134 | CH₂OCO(2,6-di-Cl-Ph) |
| 134 | CH₂O(2,4,6-triF-Ph) |
| 136 | CH₂O(2,3,5,6-tetraF-Ph) |
| 137 | CH₂OPOPh₂ |
| 138 | CH₂OPO(Me)Ph |

### EXAMPLES 139-141

Starting from N-(valinyl)aspartic acid, β-tert-butyl, α-methyl ester (see Example 36, Part B), following the general methods described in Example 46, Parts C through G and utilizing (2-naphthyl)acetic acid in place of (1-naphthyl)acetic acid in Part C, and the appropriate acid or phenol in place of 3-hydroxy-1,2,3-benzotriazin-4(3H)-one in Part F, the compounds shown below in Table 5 may also be prepared:

TABLE 5

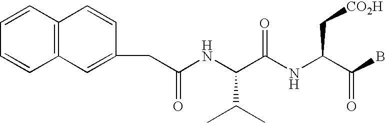

| Ex. | B |
|---|---|
| 139 | CH₂OCO(2,6-di-Cl-Ph) |
| 140 | CH₂O(2,4,6-triF-Ph) |
| 141 | CH₂O(2,3,5,6-tetraF-Ph) |

### EXAMPLES 142-143

Starting from N-(valinyl)aspartic acid, β-tert-butyl, α-methyl ester (see Example 46, Part B), following the general methods described in Example 46, Parts C through G and utilizing 3-(1-naphthyl)propionic acid in place of (1-naphthyl)acetic acid in Part C, and the appropriate acid or phenol in place of 3-hydroxy-1,2,3-benzotriazin-4(3H)-one in Part F, the compounds shown below in Table 6 may also be prepared:

TABLE 6

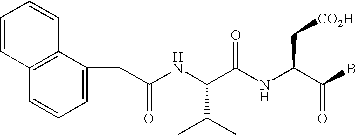

| Ex. | B |
|---|---|
| 142 | CH₂OCO(2,6-di-Cl-Ph) |
| 143 | CH₂O(1-Ph-5-CF₃-pyrazol-3-yl |

### EXAMPLE 144

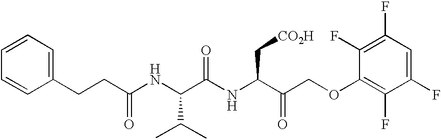

(3S)-3-[N-(3′-(Phenyl)Propionyl)Valinyl]Amino-5-(2,3,5,6-Tetrafluorophenoxy)-4-Oxopentanoic Acid

Starting from N-(valinyl)aspartic acid, β-tert-butyl, α-methyl ester (see Example 46, Part B), following the general methods described in Example 46, Parts C through G and utilizing 3-(phenyl)propionic acid in place of (1-naphthyl)acetic acid in Part C, and 2,3,5,6-tetrafluorophenol in place of 3-hydroxy-1,2,3-benzotriazin-4(3H)-one in Part F, the title compound may also be prepared.

### EXAMPLE 145

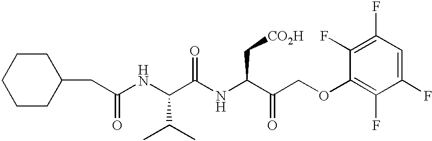

(3S)-3-[N-(CyclohexylAcetyl)Valinyl]Amino-5-(2,3,5,6-Tetrafluorophenoxy)-4-Oxopentanoic Acid

Starting from N-(valinyl)aspartic acid, β-tert-butyl, α-methyl ester (see Example 46, Part B), following the general methods described in Example 46, Parts C through G and utilizing cyclohexyl acetic acid in place of (1-naphthyl)acetic acid in Part C, and 2,3,5,6-tetrafluorophenol in place of 3-hydroxy-1,2,3-benzotriazin-4(3H)-one in Part F, the title compound may also be prepared.

### EXAMPLE 146

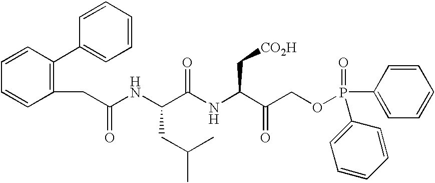

(3S)-3-[N-((2-Phenylphenyl)Acetyl)Leucinyl]Amino-5-(Diphenylphosphinyloxy)-4-Oxopentanoic Acid

Part A: [(N-Benzyloxycarbonyl)Leucinyl]Aspartic Acid, β-tert-Butyl, α-Methyl Ester

To a solution of (N-benzyloxycarbonyl)leucine, N-hydroxysuccinimide ester (4.54 g, 12.5 mmol) and aspartic acid, β-tert-butyl, α-methyl ester hydrochloride (3.00 g, 12.5 mmol) in CH₂Cl₂ (20 mL) at room temperature under nitrogen is added N-methylmorpholine (1.65 mL, 15 mmol). After stirring at room temperature for 18 hrs, the mixture is partitioned between EtOAc-water. The organic phase is washed with 5% KHSO₄, saturated NaHCO₃ and saturated NaCl solutions, dried over anhydrous Na₂SO₄ and evaporated to give the title compound.

Part B: (3S)-3-[N-((2-Phenylphenyl)Acetyl)Leucinyl]Amino-5-(Diphenylphosphinyloxy)-4-Oxopentanoic Acid

Starting with [(N-benzyloxycarbonyl)leucinyl]aspartic acid, β-tert-butyl, α-methyl ester and following the methods described in Example 55, Parts B through G, utilizing (2-phenylphenyl)acetic acid in place of (1-naphthyl)acetic acid in Part C, and the diphenylphosphinic acid in place of 3-hydroxy-1,2,3-benzotriazin-4(3H)-one in Part F, the title compound is also prepared.

### EXAMPLES 147-149

Starting with [(N-benzyloxycarbonyl)leucinyl]aspartic acid, β-tert-butyl, α-methyl ester (see Example 146, Part A) and following the methods described in Example 46, Parts B through G, utilizing (2-phenylphenyl)acetic acid in place of (1-naphthyl)acetic acid in Part C, and the appropriate acid or phenol in place of 3-hydroxy-1,2,3-benzotriazin-4(3H)-one in Part F, the compounds shown in Table 7 may also be prepared.

TABLE 7

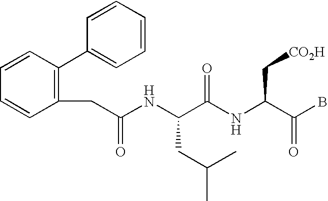

| Ex. | B |
|---|---|
| 147 | CH₂OCO(2,6-di-Cl-Ph) |
| 148 | CH₂O(2,4,6-triF-Ph) |
| 149 | CH₂O(2,3,5,6-tetraF-Ph) |

### EXAMPLE 150

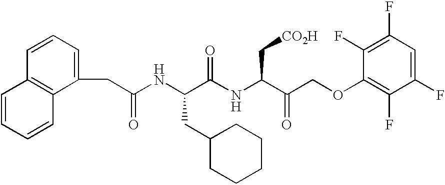

(3RS)-3-[N-((1′-Naphthyl)Acetyl)Cyclohexylalaninyl]Amino-5-(2′,3′,5′,6′-Tetrafluorophenoxy)-4-Oxopentanoic Acid

Part A: (3S)-3-(N-Benzyloxycarbonyl)Amino-5-Bromo-4-Oxopentanoic Acid tert-Butyl Ester

A solution of (N-benzyloxycarbonyl)aspartic acid, β-tert-butyl ester (2.28 g, 7.06 mmol) and N-methylmorpholine (0.85 mL, 7.7 mmol) in tetrahydrofuran (40 mL) at −10° C. (NaCl/ice bath) under nitrogen is treated dropwise via syringe with isobutyl chloroformate (1.1 mL, 8.5 mmol). After stirring at −10° C. for 20 min, the mixture is filtered (sinctered glass) into a pre-cooled receiver (ice bath) washing the filter cake with additional tetrahydrofuran (approx. 10 mL). The combined filtrate is treated with excess diazomethane/Et₂O solution (prepared from 3.10 g, 21 mmol of 1-methyl-3-nitro-1-nitrosoguanidine, 20 mL 40% KOH/10 ml Et₂O) at 0° C. (ice bath) under nitrogen. After stirring at 0° C. for 15 min and at room temperature for 30 min, the reaction mixture is again cooled to 0° C. and treated with 48% HBr(2.0 mL, 12 mmol)/acetic acid(2.0 mL). After stirring at 0° C. for 15 min and at room temperature for 15 min, the mixture is partitioned between EtOAc-water. The organic phase is washed with water, saturated NaHCO₃, and saturated NaCl solutions dried over anhydrous Na₂SO₄ and evaporated to a dryness. Trituration with hexane gives the crude title compound.

Part B: (3S,4RS)-3-(N-Benzyloxycarbonyl)Amino-5-(2′,3′,5′,6′-Tetrafluorophenoxy)-4-Hydroxypentanoic Acid tert-Butyl Ester

To a solution of (3S)-3-(N-benzyloxycarbonyl)amino-5-bromo-4-oxopentanoic acid tert-butyl ester (0.857 g, 2.14 mmol) and 2,3,5,6-tetrafluorophenol (0.410 g, 2.45 mmol) in dimethylformamide (5.0 mL) at room temperature under nitrogen is added potassium fluoride (0.40 g, 6.9 mmol). After stirring at room temperature for 16 hrs, the mixture is diluted with EtOAc, washed with saturated NaHCO₃ and saturated NaCl solutions, dried over anhydrous Na₂SO₄ and evaporated to give the crude tetrafluorophenoxymethyl ketone.

To a solution of the above crude ketone (ca 2.14 mmol) in ethanol (10 mL) at O° C. under nitrogen is added sodium borohydride (0.057 g, 1.5 mmol). After stirring at 0° C. for 1 hrs, the excess reducing agent is discharged by treatment with acetone (1.0 mL), the mixture concentrated and the residue partitioned between EtOAc-half saturated NH₄Cl solution. The organic phase is washed with saturated NaHCO₃ and saturated NaCl solutions, dried over anhydrous Na₂SO₄ and evaporated to a dryness. The residue is purified by flash chromatography on silica gel eluting with EtOAc-hexane (1:3) to give the title compound.

Part C: (3S,4RS)-3-[(N-9-Fluorenylmethoxycarbonyl)Cyclohexylalaninyl]Amino-5-(2′,3′,5′,6′-Tetrafluorophenoxy)-4-Hydroxypentanoic Acid tert-Butyl Ester

To a solution of (3S,4RS)-3-(N-benzyloxycarbonyl)amino-5-(2′,3′,5′,6′-tetrafluorophenoxy)-4-hydroxypentanoic acid tert-butyl ester (1.012 g, 2.08 mmol) in MeOH (25 mL) is added 10% Pd—C (0.30 g) and resulting mixture stirred under a hydrogen atmosphere (balloon) for 4 hrs. The mixture is filtered through Celite washing the filter cake with CH₂Cl₂ and the combined filtrates evaporated to give the crude amine.

To a solution of (N-9-fluorenylmethoxycarbonyl)cyclohexylalanine (0.763 g, 1.94 mmol) in CH₂Cl₂(10 mL) at 0° C. (ice bath) under nitrogen is added hydroxybenzotriazole hydrate (0.282 g) followed by 1-ethyl-3-(3′,3′-dimethyl-1′-aminopropyl)carbodiimide hydrochloride (0.447 g, 2.33 mmol). After stirring at 0° C. for 10 min, the mixture is treated with the above crude amine (0.682 g, ca 1.93 mmol) and the reacton allowed to warm to room temperature. After stirring at room temperature for 3 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The residue is purified by flash chromatography eluting with EtOAc-hexane (1:2) to give the title compound.

Part D: (3S,4RS)-3-[Cyclohexylalaninyl]Amino-5-(2′,3′,5′,6′-Tetrafluorophenoxy)-4-Hydroxypentanoic Acid tert-Butyl Ester

A mixture of (3S,4RS)-3-[(N-9-fluorenylmethoxycarbonyl)cyclohexylalaninyl]amino-5-(2′,3′,5′,6′-tetrafluorophenoxy)-4-hydroxypentanoic acid tert-butyl ester (1.028 g, 1.4 mmol) and 10% piperidine/dimethylformamide (3.0 mL) is stirred at room temperature under nitrogen for 2 hrs. The mixture is diluted with CH₂Cl₂, washed with water and saturated NaHCO₃ solution, dried over anhydrous anhydrous Na₂SO₄ and evaporated to dryness. The residue is purified by flash chromatography eluting with isopropanol-CH₂Cl₂ (7:93) to give the title compound.

Part E: (3S,4RS)-3-[N-((1′-Naphthyl)Acetyl)Cyclohexylalaninyl]-Amino-5-(2′,3′,5′,6′-Tetrafluorophenoxy)-4-Hydroxypentanoic Acid tert-Butyl Ester

To a solution of (1-naphthyl)acetic acid (0.20 mmol) and (3S,4RS)-3-[cyclohexylalaninyl]amino-5-(2′,3′,5′,6′-tetrafluorophenoxy)-4-hydroxypentanoic acid tert-butyl ester (0.092 g, 0.18 mmol) in CH₂Cl₂(5.0 mL) at 0° C. (ice bath) under nitrogen is added hydroxybenzotriazole hydrate (0.050 g) followed by 1-ethyl-3-(3′,3′-dimethyl-1′-aminopropyl)carbodiimide hydrochloride (0.042 g, 0.22 mmol). After stirring at 0° C. for 10 min and at room temperature for 18 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to give the crude title compound.

Part F: (3RS)-3-[N-((1′-Naphthyl)Acetyl)Cyclohexylalaninyl]-Amino-5-(2′,3′,5′,6′-Tetrafluorophenoxy)-4-Oxoxypentanoic Acid tert-Butyl Ester

To a solution of crude (3S,4RS)-3-[N-((1′-naphthyl)-acetyl)-cyclohexylalaninyl]amino-5-(2′,3′,5′,6′-tetrafluorophenoxy)-4-hydroxypentanoic acid tert-butyl ester (ca 0.18 mmol) in CH₂Cl₂ (5 mL) at room temperature under nitrogen is added Dess-Martin Periodinane (0.099 g, 0.23 mmol). After stirring at room temperature for 1.5 hrs, the mixture is diluted with EtOAc, washed with 1.0 M Na₂S₂O₃, saturated NaHCO₃ and saturated NaCl solutions, dried over anhydrous Na₂SO₄ and evaporated to a dryness. The residue is purified by flash chromatography on silica gel eluting with EtOAc-CH₂Cl₂-hexane (1:1:2) to give the title compound. Racemization of the center alpha to the ketone may occur at some point in the synthesis.

Part G: (3RS)-3-[N-((1′-Naphthyl)Acetyl)Cyclohexylalaninyl]-Amino-5-(2′,3′,5′,6′-Tetrafluorophenoxy)-4-Oxoxypentanoic Acid

To a solution of (3RS)-3-[N-((1′-naphthyl)acetyl)-cyclohexylalaninyl]-amino-5-(2′,3′,5′,6′-tetrafluorophenoxy)-4-oxopentanoic acid, tert-butyl ester (0.125 mmol) in CH₂Cl₂(2.0 mL) at room temperature under nitrogen is added trifluoroacetic acid (1.0 mL). The resulting clear solution is stirred at room temperature for 1 hrs, evaporated to dryness and chased with toluene-CH₂Cl₂ (1:1) to give the title compound.

### EXAMPLES 151-153

Starting with (3S,4RS)-3-[cyclohexylalaninyl]amino-5-(2′,3′,5′,6′-tetrafluorophenoxy)-4-hydroxypentanoic acid tert-butyl ester (see Example 150, Part D) and following the methods described in Example 150, Parts E through G, the compounds shown below in Table 8 may also be prepared:

TABLE 8

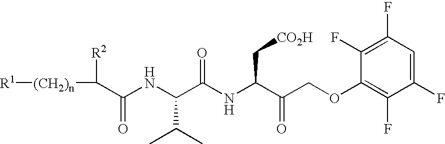

| Ex. | R¹ | n | R² |
|---|---|---|---|
| 151 | 2-naphthyl | 0 | H |
| 152 | 1-naphthyl | 1 | H |
| 153 | (2-Ph)Ph | 0 | H |

### EXAMPLE 154-155

Starting from (M-benzyloxycarbonyl)alanine and following the general methods described in Example 46, Parts A through G. utilizing either (2-phenylphenyl)acetic acid or (2-naphthyl)acetic acid in place of (1 -naphthyl)acetic acid in Part C, and 2,3,5,6-tetrafluorophenol in place of 3-hydroxy-1,2,3-benzotriazin-4(3H)-one in Part F, the compounds shown below in Table 9 may also be prepared.

TABLE 9

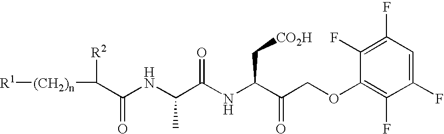

| Ex. | R¹ | n | R² |
|---|---|---|---|
| 154 | 2-naphthyl | 0 | H |
| 155 | (2-Ph)Ph | 0 | H |

### EXAMPLE 156

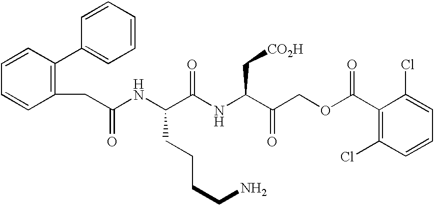

(3S)-3-[N-α-((2′-Phenylphenyl)Acetyl)Lysinyl]Amino-5-(2′,6′-Dichlorobenzoyloxy)-4-Oxopentanoic Acid Trifluoroacetate Salt

Starting from (N-α-benzyloxycarbonyl-N-ε-t-butoxycarbonyl)lysine and following the general methods described in Example 46, Parts A through G, utilizing (2-phenylphenyl)acetic acid in place of (1-naphthyl)acetic acid in Part C, and 2,6-dichlorobenzoic acid in place of 3-hydroxy-1,2,3-benzotriazin-4(3H)-one in Part F, the title compound is also prepared.

### EXAMPLE 157

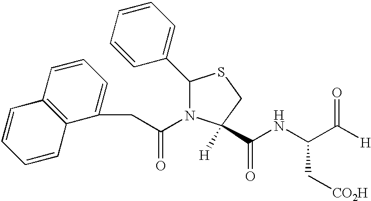

(3S,2′RS,4′R)-3-[3′-((1-Naphthyl)Acetyl)-2′-Phenylthiazolidine-4′-Carbonyl]Amino-4-Oxobutanoic Acid

Part A: (2RS,4R)-2-Phenylthiazolidine-4-Carboxylic Acid, Methyl Ester

To a suspension of L-cysteine methyl ester hydrochloride (1.717 g, 10 mmol) in tetrahydrofuran (5.0 mL) at room temperature under nitrogen is added benzaldehyde (1.02 mL, 10 mmol) followed by triethylamine (4.2 mL, 30 mmol). After stirring at room temperature for 3.5 hrs, the resulting mixture is filtered through a pad of silica gel eluting with EtOAc. Evaporation of the filtrate gives the title compound.

Part B: (2RS,4R)-3-((1-Naphthyl)Acetyl)-2-Phenylthiazolidine-4-Carboxylic Acid, Methyl Ester

To a solution of (1-naphthyl)acetic acid (15 mmol) and pyridine (1.46 mL, 18 mmol) in CH₂Cl₂ (50 mL) at room temperature under nitrogen is added cyanuric fluoride (1.52 mL, 18 mmol). After stirring at room temperature for 3 hrs, the mixture is filtered through sinctered glass and the filtrate evaporated to a viscous oil. The residue is taken up in CH₂Cl₂ and diluted with CH₂Cl₂ to a total volume of 15.0 mL (ca 1.0 mmol/ml).

To a solution of (2RS,4R)-2-phenylthiazolidine-4-carboxylic Acid, methyl ester (1.953 g, 8.7 mmol) and 2,6-di-tert-butylpyridine (1.95 mL, 8.7 mmol) in CH₂Cl₂ (22 mL) at −30° C. (dry ice/acetonitrile bath) under nitrogen is added the above acid fluoride solution (9.0 mL, ca 9.0 mmol). After stirring at −30° C. for 6 hrs, the mixture is allowed to slowly warm to room temperature. After stirring at room temperature for 16 hrs, the mixture is concentrated and the residue partitioned between EtOAc-water. The EtOAc extract is washed with water, 5% KHSO₄, saturated NaHCO₃ and saturated NaCl solutions, dried over anhydrous Na₂SO₄ and evaporated to dryness. The residue is purified by flash chromatography on silica gel eluting with EtOAc-hexane (1:3) to give the title compound.

Part C: (2RS,4R)-3-((1-Naphthyl)Acetyl)-2-Phenylthiazolidine-4-Carboxylic Acid

To a solution of (2RS,4R)-3-((1-naphthyl)acetyl)-2-phenylthiazolidine-4-carboxylic acid, methyl ester (6.14 mmol) in dioxane(15 mL)-water(5.0 mL) at room temperature is added 1.0 N LiOH solution (6.75 mL, 6.75 mmol). After stirring at room temperature for 16 hrs, the mixture is partitioned between EtOAc-5% KHSO₄. The organic phase is washed with saturated NaCl solution, dried over anhydrous Na₂SO₄ and evaporated to give the title compound.

Part D: (3S,2′RS,4′R)-3-[3′-((1-Naphthyl)Acetyl)-2′-Phenylthiazolidine-4′-Carbonyl]Amino-4-Oxobutanoic Acid tert-Butyl Ester Semicarbazone

To a solution of (2RS,4R)-3-((1-naphthyl)acetyl)-2-phenylthiazolidine-4-carboxylic acid (1.00 mmol) in CH₂Cl₂(10 mL) at 0° C. (ice bath) under nitrogen is added hydroxybenzotriazole hydrate (0.161 g) followed by 1-ethyl-3-(3′,3′-dimethyl-1′-aminopropyl)carbodiimide hydrochloride (0.288 g, 1.50 mmol). After stirring at 0° C. for 10 min, (3S)-amino-4-oxobutanoic acid tert-butyl ester semicarbazone, p-toluenesulfonate salt (0.402 g, 1.0 mmol) followed by N-methylmorpholine (0.12 mL, 1.0 mmol) is added. After stirring at 0° C. for 2 hrs and at room temperature for 18 hrs, the mixture is partitioned between EtOAc-water. The organic phase is washed with water, 5% KHSO₄, saturated NaHCO₃ and saturated NaCl solutions, dried over anhydrous Na₂SO₄ and evaporated to dryness. The crude product is purified by flash chromatography eluting with EtOAc to give the title compound.

Part E: (3S,2′RS,4′R)-3-[3′-((1-Naphthyl)Acetyl)-2′-Phenylthiazolidine-4′-Carbonyl]Amino-4-Oxobutanoic Acid Semicarbazone

To a solution (3S,2′RS ,4′R)-3-[3′-((1-naphthyl)acetyl)-2′-phenylthiazolidine-4′-carbonyl]amino-4-oxobutanoic acid tert-butyl ester semicarbazone (0.40 mmol) in CH₂Cl₂(2.6 mL)-anisole(0.1 mL) at room temperature under nitrogen is added trifluoroacetic acid (0.61 mL). The resulting solution is stirred at room temperature for 18 hrs, evaporated to dryness and chased with toluene-CH₂Cl₂ (1:1). The residue is triturated with Et₂O to give the title compound.

Part F: (3S,2′RS,4′R)-3-[3′-((1-Naphthyl)Acetyl)-2′-Phenylthiazolidine-4′-Carbonyl]Amino-4-Oxobutanoic Acid

A solution of (3S,2′RS ,4′R)-3-[3′-((1-naphthyl)acetyl)-2′-phenylthiazolidine-4′-carbonyl]amino-4-oxobutanoic acid semicarbazone (0.355 mmol) in 37% aqueous formaldehyde-acetic acid-methanol (1:1:3; v:v:v; 7.0 mL) is stirred at room temperature under nitrogen for 18 hrs. The resulting solution is concentrated on a rotovap, diluted with water, frozen and lyophilized. The residue is taken up in MeOH, filtered through Celite and evaporated to dryness. The residue is triturated with Et₂O to give the title compound.

### EXAMPLES 158-162

Following the general methods described in Example 157, Parts A through F, utilizing the appropiate aldehyde in place of benzaldehyde in Part A, the compounds shown in Table 10 may also be prepared. In the case of Example 162, (4R)-thiazolidine-4-carboxylic acid, methyl ester is prepared by treatment of (4R)-thiazolidine-4-carboxylic acid (Sigma) with HCl(g) in MeOH.

TABLE 10

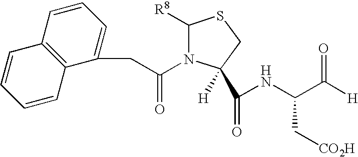

| Ex. | R⁸ |
|---|---|
| 158 | n-propyl |
| 159 | n-hexyl |
| 160 | iso-propyl |
| 161 | cyclo-hexyl |
| 162 | H |

### EXAMPLE 163

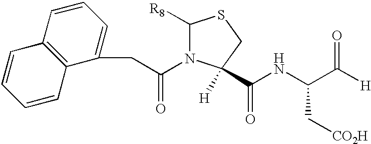

(3S)-3-[N-((1-Naphthyl)Acetyl)-4′(trans)-Hydroxyprolinyl]Amino-4-Oxobutanoic Acid

Part A: N-((1-Naphthyl)Acetyl)-4′(trans)-Hydroxyproline, Methyl Ester

To a solution of (1-naphthyl)acetic acid (9.23 mmol) and 4(trans)-hydroxyproline, methyl ester (1.34 g, 9.23 mmol) in CH₂Cl₂(92 mL) at 0° C. (ice bath) under nitrogen is added hydroxybenzotriazole hydrate (1.48 g) followed by 1-ethyl-3-(3′,3′-dimethyl-1′-aminopropyl)carbodiimide hydrochloride (2.65 g, 13.8 mmol). After stirring at 0° C. for 1 hrs and at room temperature for 6 hrs, the mixture is concentrated and the residue partitioned between EtOAc-water. The organic phase is washed with water, 5% KHSO₄, saturated NaHCO₃ and saturated NaCl solutions, dried over anhydrous Na₂SO₄ and evaporated to give the title compound.

Part B: (3S)-3-[N-((1-Naphthyl)Acetyl)-4′(trans)-Hydroxyprolinyl]Amino-4-Oxobutanoic Acid

Starting with N-((1-naphthyl)acetyl)-4′(trans)-hydroxyproline, methyl ester and following the general methods described in Example 157, Parts C through F, the title compound is also prepared.

### EXAMPLE 164

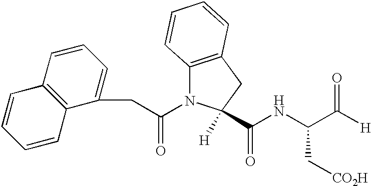

(2′S,3S)-3-[N-((1-Naphthyl)Acetyl)Indoline-2′-Carbonyl]Amino-4-Oxobutanoic Acid

Starting with (2S)—N-[(1-naphthyl)acetyl]indoline-2-carboxylic acid (see Example 55, Part B) and following the general methods described in Example 166, Parts D through F, the title compound is also prepared.

### EXAMPLE 165

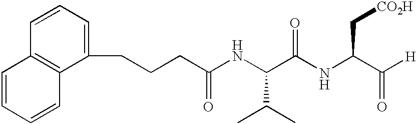

(3S)-3-[N-(4-(1′-Naphthyl)Butyryl)Valinyl]Amino-4-Oxobutanoic Acid

Part A: (3S)-3-[N-(9-Fluorenylmethoxycabonyl)Valinyl]Amino-4-Oxobutanoic Acid (tert-Butyl) Ester Semicarbazonyl-4-[2′-(4-Ethyl-Phenoxyacetyl)]Aminomethylpolystrene

Aminomethylpolystryene resin (10.0 g, 100-200 mesh, 0.71 meq/g) is placed in a 200 mL filter tube equipped with a vacuum stopcock and glass frit and washed successively with CH₂Cl₂(50 mL)/dimethylformamide(50 mL), diisopropylethylamine(5 mL)/dimethylformamide(30 mL), dimethylformamide (2×50 mL) and tetrahydrofuran (30 mL). The resin is suspended in tetrahydrofuran(20 mL)/N-methylpyrolidinone(20 mL) with nitrogen agitation through the bottom of the frit and treated with diiospropylethylamine (1.9 mL, 10.9 mmol) and (3S)-3-(9-fluorenylmethoxycabonyl)amino-4-oxobutanoic acid (tert-butyl) ester semicarbazonyl-4-[2′-(4-ethyl-phenoxyacetic acid)] (2.24 g, 3.56 mmol). After all of the solid has dissolved (approx. 10 min), the mixture is treated with pyBOP [benzotriazolyloxy-tris(N-pyrolidinyl)phosphonium hexafluorophosphate, 2.78 g, 5.34 mmol) in one portion. After mixing by nitrogen agitation for 3 hrs, the supernatant is removed by suction and the resin washed succesively with tetrahydrofuran (2×50 mL), dimethylformamide (3×50 mL) and CH₂Cl₂ (2×50 mL). Unreacted amine groups are capped by treatment with a mixture of acetic anhydride(10 mL)/ dimethylformamide(30 mL)/diisopropylethylamine(1.0 mL). After mixing by nitrogen agitation for 1 hrs, the supernatant is removed by suction and the resin washed with dimethylformamide(4×50 mL).

The resin is treated with piperidine(10 mL)/ dimethylformamide(40 mL) and mixed by nitrogen agitation for 1 hrs. The supernatant is removed by suction and the resin washed with dimethylformamide(4×50 mL) and tetrahydrofuran (50 mL).

The resin is suspended in tetrahydrofuran(20 mL)/N-methylpyrolidinone(20 mL), treated with N-(9-fluorenylmethoxycabonyl)valine (3.63 g, 10.7 mmol), diisopropylethylamine (5.7 mL, 32.7 mmol) and pyBOP (8.34 g, 16.0 mmol) and mixed by nitrogen agitation for 2.5 hrs. The supernatant is removed by suction and the resin washed succesively with dimethylformamide (3×40 mL) and CH₂Cl₂ (3×40 mL), methanol (2×40 mL) and Et₂O (2×40 mL). The resin is dried in vacuo to give the title product. Based on the starting semicarbazone-acid, the resin loading may be calculated as approximately 0.28 meq/g.

Part B: (3S)-3-[N-(4-(1′-Naphthyl)Butyryl)Valinyl]Amino-4-Oxobutanoic Acid

An aliquot of the Part A resin (0.125 g, ca 0.035 mmol) is placed in a 6 mL Supelco™ fitration tube equipped with a 20 μm polyethylene frit, treated with piperidine-dimethylformamide (1.0 mL, 1:4 v/v) and mixed on an orbital shaker for 1 hrs. The supernatant is removed by suction and the resin washed with dimethylformamide (4×1.0 mL) and CH₂Cl₂ (3×1.0 mL). The resin is treated with 0.5M iPr₂NEt in N-methylpyrolidinone (0.40 mL, 0.20 mmol), 4-(1-naphthyl)butyric acid (0.115 mmol) and 0.25M O-(7-azabenzotriazol-1-yl)-N,N,N′,N′-tetramethyluronium hexafluorophate in N-methylpyrolidinone (0.40 mL, 0.10 mmol). The mixture is mixed on an orbital shaker under an nitrogen atmosphere for 16 hrs. The supernatant is removed by suction and the resin washed succesively with dimethylformamide (3×1.0 mL) and CH₂Cl₂ (3×1.0 mL), methanol (2×1.0 mL) and Et₂O (2×1.0 mL).

The resin is treated with 1.0 mL of CH₂Cl₂ and allowed to re-swell for 15 min. The solvent is removed by suction and the resin treated with trifluoroacetic acid-CH₂Cl₂-anisole (1.0 mL, 4:3:1 v/v/v). After mixing on an orbital shaker under nitrogen for 5.5 hrs, the supernatant is removed by suction and the resin washed with CH₂Cl₂ (4×1.0 mL). The resin is treated with 37% aqueous formaldehyde-acetic acid-tetrahydrofuran-trifluoroacetic acid (1.0 mL, 1:1:5:0.025 v/v/v/v) and mixed on an orbital shaker under nitrogen for 4.5 hrs. The supernatant is collected by suction, the resin washed with tetrahydrofuran (3×0.5 mL). The combined filtrates are blown down under nitrogen. The residue is taken up in methanol (0.5 mL), filtered and applied directly to a 3 mL Supelco™ LC-18 reverse phase extraction tube which has been pre-conditioned with water, and eluted successively with 3 mL each of 10% MeOH-water, 30% MeOH-water, 60% MeOH-water and 90% MeOH-water. The product-containing fractions (TLC) are combined and evaporated to dryness to give the title compound.

### EXAMPLES 166-170

Starting with (3S)-3-[N-(9-fluorenylmethoxycabonyl)valinyl]amino-4-oxobutanoic acid (tert-butyl) ester semicarbazonyl-4-[2′-(4-ethyl-phenoxyacetyl)]aminomethylpolystrene (see Example 165, Part A) and following the methods described in Example 165, Part B, the compounds shown below in Table 11 may also be prepared:

TABLE 11

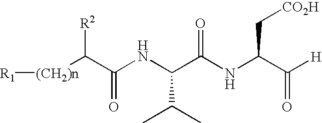

| Ex. | R¹ | n | R² |
|---|---|---|---|
| 166 | (2-t-Bu)Ph | 0 | H |
| 167 | (2-Ph)Ph | 0 | H |
| 168 | (2-Ph)Ph | 0 | CH₃ |
| 169 | (2-Ph)Ph | 1 | H |
| 170 | 1-naphthyl | 1 | H |

Although the invention has been described with reference to the examples provided above, it should be understood that various modifications can be made without departing from the spirit of the invention. Accordingly, the invention is limited only by the claims.

We claim:

1. A compound of the following formula: 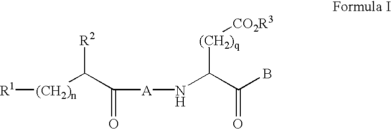
wherein:
n is 0, 1 or 2;
q is 1 or 2;
A is a natural or unnatural amino acid of Formula IIa-i: 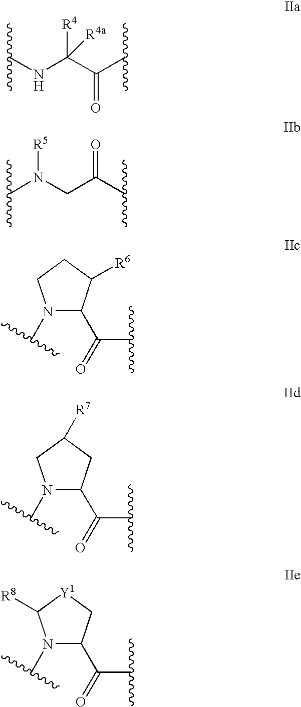 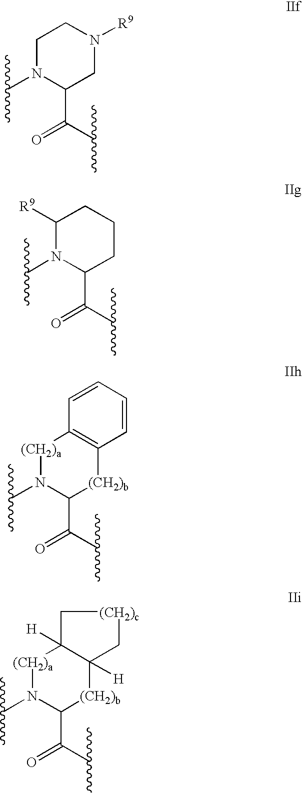
B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₘcycloalkyl, (CH₂)ₘphenyl, (CH₂)ₘ(substituted phenyl), (CH₂)ₘ(1 or 2-naphthyl), (CH₂)ₘheteroaryl, halomethyl, CO₂R¹³, CONR¹⁴R¹⁵, CH₂ZR¹⁶, CH₂OCO(aryl), CH₂OCO(substituted aryl), CH₂OCO(heteroaryl), CH₂OCO(substituted heteroaryl), or CH₂OPO(R¹⁷)R¹⁸, where Z is an oxygen or a sulfur atom, or B is a group of the Formula IIa-c: 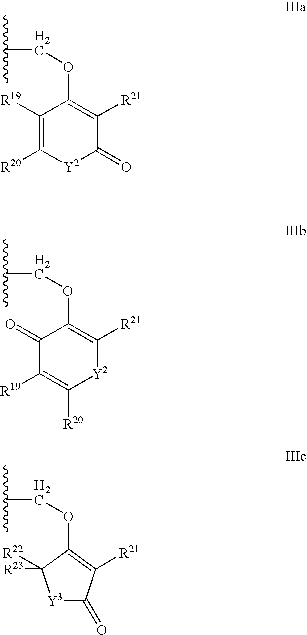
R¹ is cycloalkyl, substituted cycloalkyl, phenyl, substituted phenyl, naphthyl, substituted naphthyl, heteroaryl, or substituted heteroaryl;
R² is hydrogen, alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) pyridyl, thienyl, furyl, thiazolyl, imidazolyl, pyrazolyl, isoxazolyl, pyrazinyl, pyrimidyl, triazinyl, tetrazolyl, and indolyl;
R³ is hydrogen, alkyl, cycloalkyl, (cycloalkyl)alkyl, phenylalkyl, or substituted phenylalkyl;
and wherein
R⁴ is alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) pyridyl, thienyl, furyl, thiazolyl, imidazolyl, pyrazolyl, isoxazolyl, pyrazinyl, pyrimidyl, triazinyl, tetrazolyl, and indolyl;
R^{4a} is hydrogen, or methyl, or R⁴ and R^{4a} taken together are —(CH₂)d— where d is an interger from 2 to 6;
R⁵ is phenyl, substituted phenyl, (CH₂)ₚphenyl, (CH₂)ₚ(substituted phenyl), cycloalkyl, or benzofused cycloalkyl;
R⁶ is hydrogen, alkyl, cycloalkyl, phenyl, substituted phenyl, (CH₂)ₘcycloalkyl, (CH₂)ₘphenyl, (CH₂)ₘ(substituted phenyl), or (CH₂)ₘ(1 or 2-naphthyl);
R⁷ is hydrogen, fluorine, oxo, alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹¹, SR¹², or NHCOR¹⁰;
R⁸ is hydrogen, oxo, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);
R⁹ is alkyl, cycloalkyl, (CH₂)ₘcycloalkyl, (CH₂)ₘphenyl, (CH₂)ₘ(substituted phenyl), (CH₂)ₘ(1 or 2-naphthyl), or COR¹⁰;
R¹⁰ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹³, or NR¹⁴R¹⁵;
R¹¹ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);
R¹² is alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);
R¹³ is alkyl, cycloalkyl, (CH₂)ₘcycloalkyl, (CH₂)ₘphenyl, (CH₂)ₘ(substituted phenyl), or (CH₂)ₘ(1 or 2-naphthyl);
R¹⁴ is hydrogen, alkyl, cycloalkyl, phenyl, substituted phenyl, naphthyl, substituted naphthyl, (CH₂)ₘcycloalkyl, (CH₂)ₘphenyl, (CH₂)ₘ(substituted phenyl), or (CH₂)ₘ(1 or 2-naphthyl);
R¹⁵ is hydrogen or alkyl; or
R¹⁴ and R¹⁵ taken together form a five, six or seven membered carbocyclic or heterocyclic ring, such as morpholine or N-substituted piperazine;
R¹⁶ is phenyl, substituted phenyl, naphthyl, substituted naphthyl, heteroaryl, (CH₂)ₘphenyl, (CH₂)ₘ(substituted phenyl), (CH₂)ₘ(1 or 2-naphthyl), or (CH₂)ₘheteroaryl;
R¹⁷ and R¹⁸ are independently alkyl, cycloalkyl, phenyl, substituted phenyl, naphthyl, or phenylalkyl, substituted phenylalkyl, or (cycloalkyl)alkyl;
R¹⁹ and R²⁰ are independently hydrogen, alkyl, phenyl, substituted phenyl, (CH₂)ₘphenyl, or (CH₂)ₘ(substituted phenyl), or R19 and R²⁰ taken together are —(CH═CH)₂—;
R²¹ is hydrogen, alkyl, phenyl, substituted phenyl, (CH₂)ₘphenyl, (CH₂)ₘ(substituted phenyl);
R²², R²³ and R²⁴ are independently hydrogen or alkyl;
Y¹ is CH₂, (CH₂)₂, (CH₂)₃, or S;
Y² is O or NR²⁴;
Y³ is CH₂, O, or NR²⁴;
a is 0 or 1 and b is 1 or 2, provided that when a is 1 then b is 1;
c is 1 or 2, provided that when c is 1 then a is 0 and b is 1;
m is 1, 2, 3or 4; and
p is 1 or 2;
or a pharmaceutically acceptable salt thereof.

2. The compound of claim 1 wherein q is 1.

3. The compound of claim 1 wherein q is 2.

4. The compound of claim 1 wherein A is

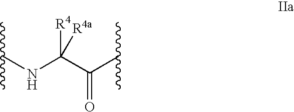

5. The compound of claim 4 wherein
R⁴ is lower alkyl, cycloalkyl, phenyl, substituted phenyl, (CH₂)ₙNH₂, (CH₂)ₘOR¹⁰, (CH₂)ₘSR¹¹, (CH₂)ₙcycloalkyl, (CH₂)ₙphenyl, (CH₂)ₙ(substituted phenyl), or (CH₂)ₘ(1 or 2-naphthyl); and
R^{4a} is hydrogen.

6. The compound of claim 1 wherein A is

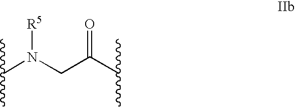

7. The compound of claim 6 wherein R⁵ is phenyl, substituted phenyl, (CH₂)ₘphenyl, (CH₂)ₘ(substituted phenyl), cycloalkyl, or 2-indanyl.

8. The compound of claim 1 wherein A is

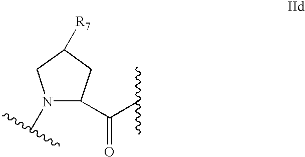

9. The compound of claim 8 wherein R⁷ is hydrogen, fluorine, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, or SR¹¹.

10. The compound of claim 1 wherein A is

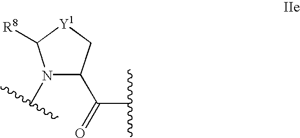

11. The compound of claim 10 wherein
R⁸ is hydrogen, oxo, cycloalkyl, phenyl, substituted phenyl, or naphthyl; and
Y¹ is CH₂, (CH₂)₂, (CH₂)₃, or S.

12. The compound of claim 1 wherein A is

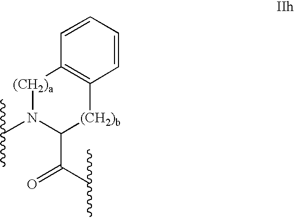

13. The compound of claim 12 wherein a is 0.

14. The compound of claim 1 wherein B is hydrogen.

15. The compound of claim 1 wherein
B is 2-benzoxazolyl, substituted 2-oxazolyl, CH₂ZR¹⁵, CH₂OCO(aryl), or CH₂OPO(R¹⁶)R¹⁷; and
Z is O or S.

16. The compound of claim 1 wherein B is

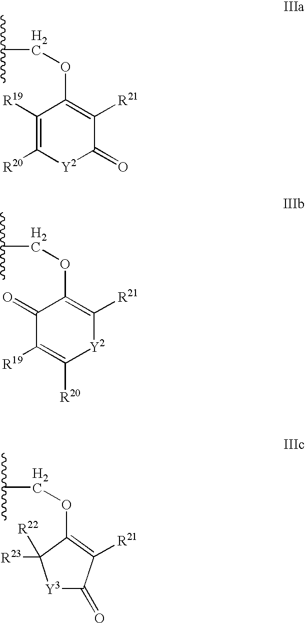

17. The compound of claim 16 wherein R¹⁹ and R²⁰ are independently hydrogen, alkyl, or phenyl, or wherein R¹⁹ and R²⁰ taken together are —(CH═CH)₂—.

18. The compound of claim 1 wherein
n is 0 or 1;
q is 1;
R¹ is substituted phenyl, naphthyl, or substituted naphthyl;
R² is hydrogen, lower alkyl, (CH₂)ₚCO₂R³, (CH₂)ₘ(substituted phenyl), (CH₂)ₘ(1- or 2-naphthyl), or (CH₂)ₘtetrazolyl; and
R³ is hydrogen or lower alkyl.

19. The compound of claim 18 wherein R¹ is 1-naphthyl.

20. The compound of claim 18 wherein R¹ is 2-naphthyl.

21. The compound of claim 18 wherein R¹ is substituted naphthyl.

22. The compound of claim 18 wherein R¹ is substituted phenyl.

23. The compound of claim 22 wherein substituted phenyl is 2-substituted phenyl.

24. The compound of claim 23 wherein 2-substituted phenyl is (2-phenyl)phenyl.

25. The compound of claim 18 wherein A is alanine, valine, leucine cyclohexylalanine, phenylgycine or t-butylglycine.

26. The compound of claim 25 wherein R¹ is 1-naphthyl.

27. The compound of claim 25 wherein R¹ is 2-naphthyl.

28. The compound of claim 25 wherein R¹ is substituted naphthyl.

29. The compound of claim 25 wherein R¹ is 2-substituted phenyl.

30. The compound of claim 29 wherein 2-substituted phenyl is (2-phenyl)phenyl.

31. The compound of claim 18 wherein R² is (CH₂)₂CO₂R³ and n is 0.

32. The compound of claim 18 wherein R² is (CH₂)ₘtetrazolyl and m is 0.

33. The compound of claim 1 wherein R³ is hydrogen.

34. The compound of claim 1 in the cyclic ketal form and having the following structure:

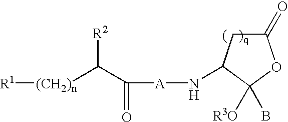

35. The compound of claim 34 wherein B is lower alkyl or benzyl.

36. A pharmaceutical composition comprising a compound of claim 1 in combination with a pharmaceutically acceptable carrier.

37. A method for treating an autoimmune disease, comprising administering an effective amount of the pharmaceutical composition of claim 36 to a patient in need thereof.

38. A method of treating an inflammatory disease, comprising administering an effective amount of the pharmaceutical composition of claim 36 to a patient in need thereof.

39. A method of treating a neurodegenerative disease, comprising administering an effective amount of the pharmaceutical composition of claim 36 to a patient in need thereof.

40. A method of preventing ischemic injury to a patient suffering from a disease associated with ischemic injury, comprising administering an effective amount of the pharmaceutical composition of claim 36 to a patient in need thereof.

41. A method for expanding of hematopoietic cell populations or enhancing their survival, comprising contacting the cells with an effective amount of the pharmaceutical composition of claim 36.

42. The method of claim 41 wherein the cell populations are granulocytes, monocytes, erthrocytes, lymphocytes or platelets for use in cell transfusions.

43. A method of prolonging the viability of an organ that has been removed from a donor or isolated cells derived from an organ for the purpose of a future transplantation procedure, comprising applying an effective amount of the pharmaceutical composition of claim to the organ or isolated cells to prolong the viability of the same as compared to untreated organ or isolated cells.

44. The method of claim 43 wherein the organ is an intact organ.

45. The method of claim 43 wherein the isolated cells are pancreatic islet cells, dopaminergic neurons, blood cells or hematopoietic cells.
